(19) United States
(12) Patent Application Publication
(10) Pub. No.: US 2024/0423969 A1
(43) Pub. Date: Dec. 26, 2024

(54) SYNERGISTIC DRUG COMBINATIONS TO TREAT CANCER

(71) Applicant: Mayo Foundation for Medical Education and Research, Rochester, MN (US)

(72) Inventors: John A. Copland, III, Ponte Vedra Beach, FL (US); Kabir Mody, Jacksonville, FL (US); Justyna J. Gleba, Jacksonville, FL (US); Tushar C. Patel, Ponte Vedra Beach, FL (US)

(73) Assignee: Mayo Foundation for Medical Education and Research, Rochester, MN (US)

(21) Appl. No.: 18/631,941

(22) Filed: Apr. 10, 2024

Related U.S. Application Data
(60) Provisional application No. 63/458,627, filed on Apr. 11, 2023.

(51) Int. Cl. A61K 31/4545; A61K 31/445; A61K 31/47; A61K 31/496; A61K 31/501; A61P 35/00

(52) U.S. Cl. CPC A61K 31/4545; A61K 31/445; A61K 31/47; A61K 31/496; A61K 31/501; A61P 35/00

(57) ABSTRACT

This document provides methods and materials for treating cancer (e.g., liver cancer such as hepatocellular carcinoma (HCC)) using one or more stearoyl CoA desaturase 1 (SCD1) polypeptide inhibitors (e.g., a selective SCD1 inhibitor (SSI)) and one or more tyrosine kinase inhibitors.

### CROSS-REFERENCE TO RELATED APPLICATIONS

This application claims the benefit of U.S. Patent Application Ser. No. 63/458,627, filed on Apr. 11, 2023. The disclosure of the prior application is considered part of, and is incorporated by reference in, the disclosure of this application.

### STATEMENT REGARDING FEDERAL FUNDING

This invention was made with government support under CA210964 awarded by the National Institutes of Health. The government has certain rights in the invention.

### TECHNICAL FIELD

This document relates to methods and materials for treating cancer. For example, this document provides methods and materials for using one or more stearoyl CoA desaturase 1 (SCD1) polypeptide inhibitors (e.g., a selective SCD1 inhibitor (SSI) such as SSI-4) and one or more tyrosine kinase inhibitors (e.g., cabozantinib and/or lenvatinib) to treat cancers that exhibit little or no response to treatment with either inhibitor alone.

### BACKGROUND

Hepatocellular carcinoma (HCC) accounts for over 90% of all liver cancers and has been recognized as a leading cause of death among patients with cirrhosis. More than 50% of HCC patients are already in an advanced stage of HCC at diagnosis, and more than 70% of HCC patients experience relapse within 5 years after starting treatment.

### SUMMARY

This document provides methods and materials for using synergistic drug combinations to treat cancer (e.g., liver cancer such as HCC) within a mammal (e.g., a human). For example, this document provides methods and materials for using one or more (e.g., one, two, three, four, or more) SCD1 polypeptide inhibitors (e.g., a SSI such as SSI-4) and one or more (e.g., one, two, three, four, or more) tyrosine kinase inhibitors (e.g., cabozantinib and/or lenvatinib) to treat cancers (e.g., cancers that exhibit little or no response to treatment with either inhibitor alone). In some cases, one or more SCD1 polypeptide inhibitors (e.g., a SSI such as SSI-4) and one or more tyrosine kinase inhibitors (e.g., cabozantinib and/or lenvatinib) can be administered to a mammal a mammal (e.g., a human) having cancer (e.g., liver cancer such as HCC) to treat the mammal.

As described herein, one or more (e.g., one, two, three, four, or more) SCD1 polypeptide inhibitors (e.g., a SSI such as SSI-4) and one or more (e.g., one, two, three, four, or more) tyrosine kinase inhibitors (e.g., cabozantinib and/or lenvatinib) can be used in combination to treat cancer (e.g., reduce the number of cancer cells within a mammal) in a synergistic manner that outperforms the use of either inhibitor type alone.

In general, one aspect of this document features methods for treating cancer in a mammal. The methods can include, or consist essentially of, administering, to a mammal having cancer, a SCD1 polypeptide inhibitor and a tyrosine kinase inhibitor, where the number of cancer cells within the mammal is reduced. The mammal can be a human. The cancer can be a liver cancer. The cancer can be a hepatocellular carcinoma. The mammal can be identified as having the cancer. The SCD1 polypeptide inhibitor can inhibit expression of the SCD1 polypeptide. The SCD1 polypeptide inhibitor can inhibit activity of the SCD1 polypeptide. The SCD1 polypeptide inhibitor can be a compound having Formula (II) or Formula (IIa):

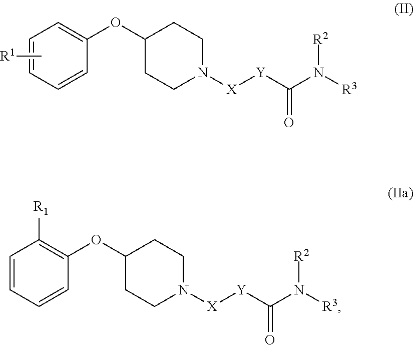

or a pharmaceutically acceptable salt thereof, where R¹ is halo; X is —(C═O)NR⁴—; Y is

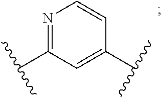 [SMILES: *c1ccnc(*)c1]

and R², R³, and R⁴ are each independently H or an unsubstituted C₁₋₆ alkyl. The SCD1 polypeptide inhibitor can be SSI-4, 2-{[4-(2-Chlorophenoxy)piperidine-1-carbonyl]amino}-N-methylpyridine-4-carboxamide:

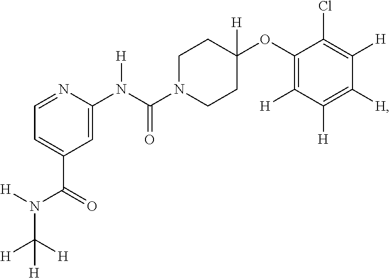

or a pharmaceutically acceptable salt thereof. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide):

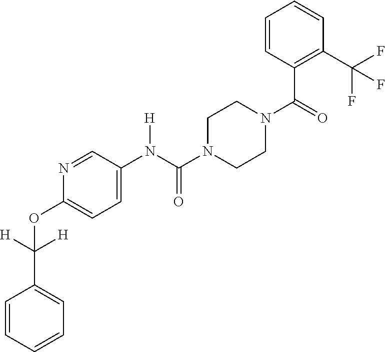 [SMILES: O=C(Nc1ccc(OCc2ccccc2)nc1)N1CCN(C(=O)c2ccccc2C(F)(F)F)CC1]

or a pharmaceutically acceptable salt thereof. The SCD1 polypeptide inhibitor can be MF-438, 2-Methyl-5-(6-(4-(2-trifluoromethyl)phenoxy)piperidin-3-yl)-1,3,4-thiadiazole):

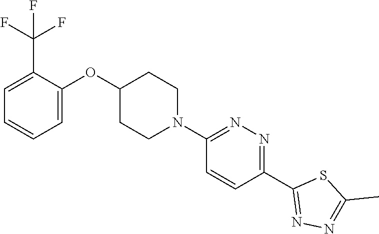 [SMILES: Cc1nnc(-c2ccc(N3CCC(Oc4ccccc4C(F)(F)F)CC3)nn2)s1]

or a pharmaceutically acceptable sale thereof. The SCD1 polypeptide inhibitor can be A939572, 4-(2-Chlorophenoxy)-N-[3-(methylcarbamoyl)phenyl]piperidine-1-carboxamide):

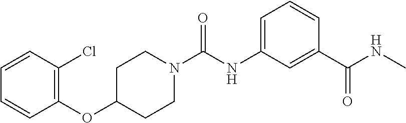 [SMILES: CNC(=O)c1cccc(NC(=O)N2CCC(Oc3ccccc3Cl)CC2)c1]

or a pharmaceutically acceptable sale thereof. The tyrosine kinase inhibitor can inhibit expression of the tyrosine kinase. The tyrosine kinase inhibitor can inhibit activity of the tyrosine kinase. The tyrosine kinase inhibitor can inhibit one or more polypeptides selected from the group consisting of a vascular endothelial growth factor receptor, a platelet-derived growth factor receptor, a fibroblast growth factor receptor, a receptor tyrosine kinase encoded by a c-KIT proto-oncogene, a receptor tyrosine kinase encoded by a RET proto-oncogene, a hepatocyte growth factor receptor, a receptor tyrosine kinase encoded by an AXL proto-oncogene, a FMS like tyrosine kinase 3, a tropomyosin receptor kinase B, and a TIE-2. The tyrosine kinase inhibitor can be cabozantinib, lenvatinib, sorafenib, sunitinib, pr regorafenib. The method can include administering two or more SCD1 polypeptide inhibitors to the mammal. The method can include administering two or more tyrosine kinase inhibitors to the mammal.

In another aspect, this document features methods for treating cancer in a mammal, where cancer cells of the cancer have resistance to treatment with a tyrosine kinase inhibitor alone. The methods can include, or consist essentially of, administering, to a mammal having cancer where cancer cells of the cancer have resistance to treatment with a tyrosine kinase inhibitor alone, a SCD1 polypeptide and the tyrosine kinase inhibitor, where the number of cancer cells within the mammal is reduced. The method can include determining that the cancer cells are resistant to treatment with the tyrosine kinase inhibitor alone. The mammal can be a human. The cancer can be a liver cancer. The cancer can be a hepatocellular carcinoma. The mammal can be identified as having the cancer. The SCD1 polypeptide inhibitor can inhibit expression of the SCD1 polypeptide. The SCD1 polypeptide inhibitor can inhibit activity of the SCD1 polypeptide. The SCD1 polypeptide inhibitor can be a compound having Formula (II) or Formula (IIa):

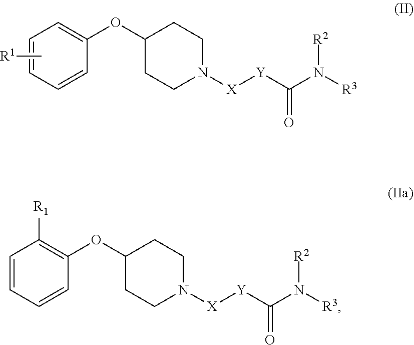

or a pharmaceutically acceptable salt thereof; where R¹ is halo; X is —(C═O)NR⁴—; Y is

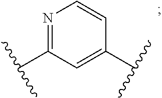 [SMILES: *c1ccnc(*)c1]

and R², R³, and R⁴ are each independently H or an unsubstituted C₁₋₆ alkyl. The SCD1 polypeptide inhibitor can be SSI-4, 2-{[4-(2-Chlorophenoxy)piperidine-1-carbonyl]amino}-N-methylpyridine-4-carboxamide:

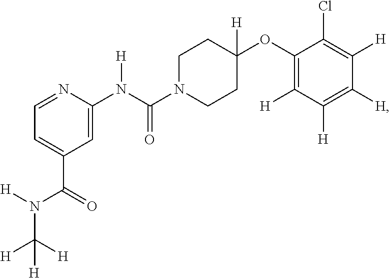

or a pharmaceutically acceptable salt thereof. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide):

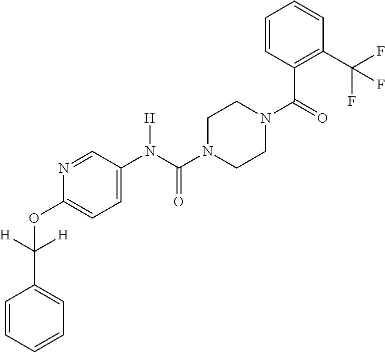 [SMILES: O=C(Nc1ccc(OCc2ccccc2)nc1)N1CCN(C(=O)c2ccccc2C(F)(F)F)CC1]

or a pharmaceutically acceptable salt thereof. The SCD1 polypeptide inhibitor can be MF-438, 2-Methyl-5-(6-(4-(2-trifluoromethyl)phenoxy)piperidin-3-yl)-1,3,4-thiadiazole):

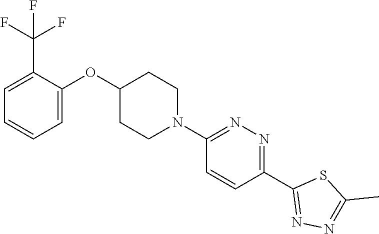 [SMILES: Cc1nnc(-c2ccc(N3CCC(Oc4ccccc4C(F)(F)F)CC3)nn2)s1]

or a pharmaceutically acceptable sale thereof. The SCD1 polypeptide inhibitor can be A939572, 4-(2-Chlorophenoxy)-N-[3-(methylcarbamoyl)phenyl]piperidine-1-carboxamide):

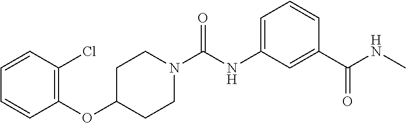 [SMILES: CNC(=O)c1cccc(NC(=O)N2CCC(Oc3ccccc3Cl)CC2)c1]

or a pharmaceutically acceptable sale thereof. The tyrosine kinase inhibitor can inhibit expression of the tyrosine kinase. The tyrosine kinase inhibitor can inhibit activity of the tyrosine kinase. The tyrosine kinase inhibitor can inhibit one or more polypeptides selected from the group consisting of a vascular endothelial growth factor receptor, a platelet-derived growth factor receptor, a fibroblast growth factor receptor, a receptor tyrosine kinase encoded by a c-KIT proto-oncogene, a receptor tyrosine kinase encoded by a RET proto-oncogene, a hepatocyte growth factor receptor, a receptor tyrosine kinase encoded by an AXL proto-oncogene, a FMS like tyrosine kinase 3, a tropomyosin receptor kinase B, and a TIE-2. The tyrosine kinase inhibitor can be cabozantinib, lenvatinib, sorafenib, sunitinib, or regorafenib. The method can include administering two or more SCD1 polypeptide inhibitors to the mammal. The method can include administering two or more tyrosine kinase inhibitors to the mammal.

In another aspect, this document features uses of a composition including a SCD1 polypeptide inhibitor and a tyrosine kinase inhibitor to treat cancer. The cancer can be a liver cancer. The cancer can be a hepatocellular carcinoma. The SCD1 polypeptide inhibitor can be a compound having Formula (II) or Formula (IIa):

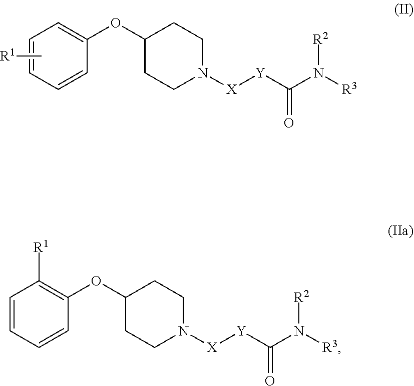

or a pharmaceutically acceptable salt thereof, where R¹ is halo; X is —(C═O)NR⁴—;

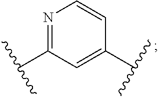 [SMILES: *c1ccnc(*)c1]

and R², R³, and R⁴ are each independently H or an unsubstituted C₁₋₆ alkyl. The SCD1 polypeptide inhibitor can be SSI-4, 2-{[4-(2-Chlorophenoxy)piperidine-1-carbonyl]amino}-N-methylpyridine-4-carboxamide:

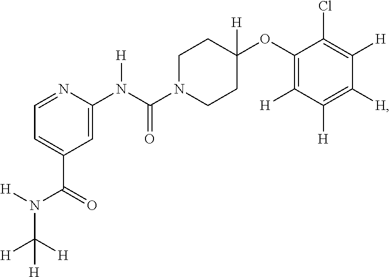

or a pharmaceutically acceptable salt thereof. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide):

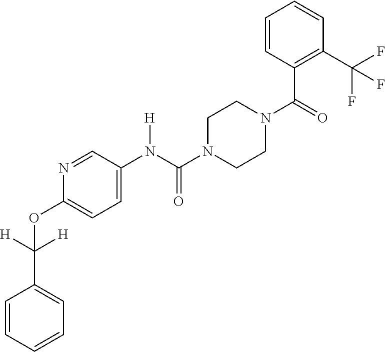 [SMILES: O=C(Nc1ccc(OCc2ccccc2)nc1)N1CCN(C(=O)c2ccccc2C(F)(F)F)CC1]

or a pharmaceutically acceptable salt thereof. The SCD1 polypeptide inhibitor can be MF-438, 2-Methyl-5-(6-(4-(2-trifluoromethyl)phenoxy)piperidin-3-yl)-1,3,4-thiadiazole):

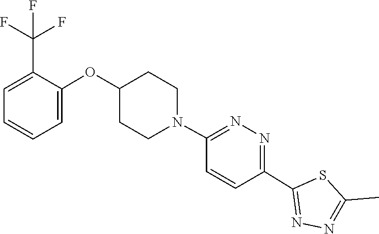 [SMILES: Cc1nnc(-c2ccc(N3CCC(Oc4ccccc4C(F)(F)F)CC3)nn2)s1]

or a pharmaceutically acceptable sale thereof. The SCD1 polypeptide inhibitor can be A939572, 4-(2-Chlorophenoxy)-N-[3-(methylcarbamoyl)phenyl]piperidine-1-carboxamide):

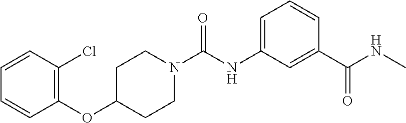 [SMILES: CNC(=O)c1cccc(NC(=O)N2CCC(Oc3ccccc3Cl)CC2)c1]

or a pharmaceutically acceptable sale thereof. The tyrosine kinase inhibitor can be cabozantinib, lenvatinib, sorafenib, sunitinib, or regorafenib. The composition can include two or more SCD1 polypeptide inhibitors. The composition can include two or more tyrosine kinase inhibitors.

In another aspect, this document features compositions including a SCD1 polypeptide inhibitor and a tyrosine kinase inhibitor for use in the preparation of a medicament to treat cancer. The cancer can be a liver cancer. The cancer can be a hepatocellular carcinoma. The SCD1 polypeptide inhibitor can be a compound having Formula (II) or Formula (IIa):

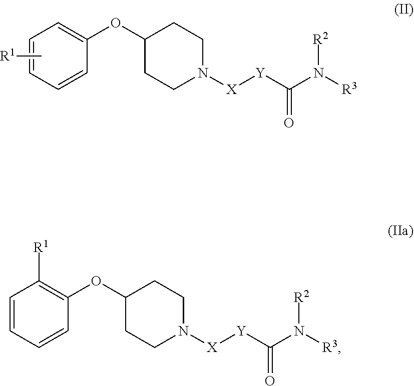

or a pharmaceutically acceptable salt thereof, where R¹ is halo; X is —(C═O)NR⁴—; Y is

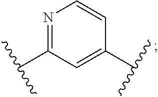 [SMILES: *c1ccnc(*)c1]

and R², R³, and R⁴ are each independently H or an unsubstituted C₁₋₆ alkyl. The SCD1 polypeptide inhibitor can be SSI-4, 2-{[4-(2-Chlorophenoxy)piperidine-1-carbonyl]amino}-N-methylpyridine-4-carboxamide:

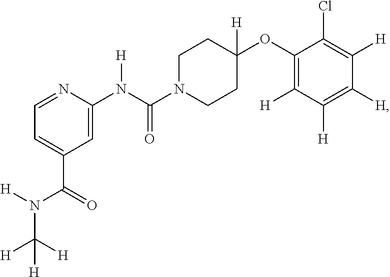

or a pharmaceutically acceptable salt thereof. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide):

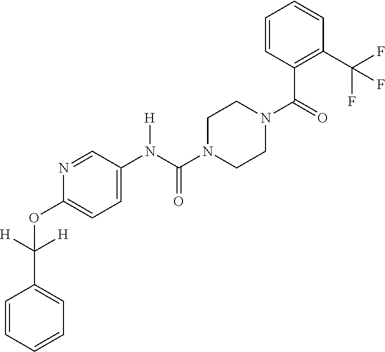 [SMILES: O=C(Nc1ccc(OCc2ccccc2)nc1)N1CCN(C(=O)c2ccccc2C(F)(F)F)CC1]

or a pharmaceutically acceptable salt thereof. The SCD1 polypeptide inhibitor can be MF-438, 2-Methyl-5-(6-(4-(2-trifluoromethyl)phenoxy)piperidin-3-yl)-1,3,4-thiadiazole):

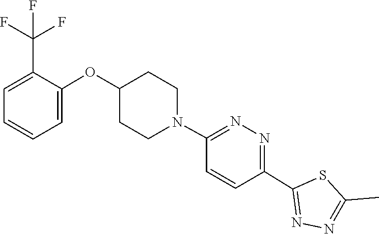 [SMILES: Cc1nnc(-c2ccc(N3CCC(Oc4ccccc4C(F)(F)F)CC3)nn2)s1]

or a pharmaceutically acceptable sale thereof. The SCD1 polypeptide inhibitor can be A939572, 4-(2-Chlorophenoxy)-N-[3-(methylcarbamoyl)phenyl]piperidine-1-carboxamide):

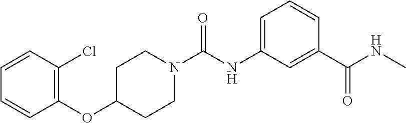 [SMILES: CNC(=O)c1cccc(NC(=O)N2CCC(Oc3ccccc3Cl)CC2)c1]

or a pharmaceutically acceptable sale thereof. The tyrosine kinase inhibitor can be cabozantinib, lenvatinib, sorafenib, sunitinib, or regorafenib. The composition can include two or more SCD1 polypeptide inhibitors. The composition can include two or more tyrosine kinase inhibitors.

In another aspect, this document features compositions including a SCD1 polypeptide inhibitor and a tyrosine kinase inhibitor for use in the treatment of cancer. The cancer can be a liver cancer. The cancer can be a hepatocellular carcinoma. The SCD1 polypeptide inhibitor can be a compound having Formula (II) or Formula (IIa):

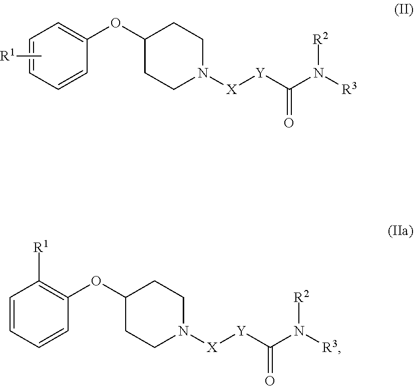

or a pharmaceutically acceptable salt thereof, where R¹ is halo; X is —(C═O)NR⁴—; Y is

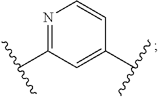 [SMILES: *c1ccnc(*)c1]

and R², R³, and R⁴ are each independently H or an unsubstituted C₁₋₆ alkyl. The SCD1 polypeptide inhibitor can be SSI-4, 2-{[4-(2-Chlorophenoxy)piperidine-1-carbonyl]amino}-N-methylpyridine-4-carboxamide:

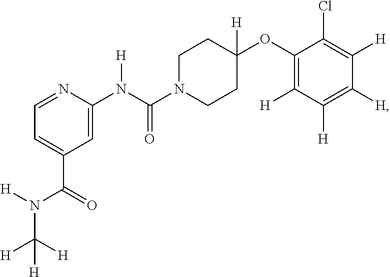

or a pharmaceutically acceptable salt thereof. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide):

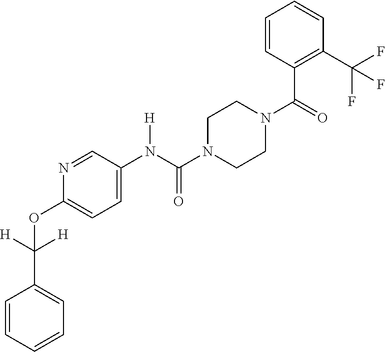 [SMILES: O=C(Nc1ccc(OCc2ccccc2)nc1)N1CCN(C(=O)c2ccccc2C(F)(F)F)CC1]

or a pharmaceutically acceptable salt thereof. The SCD1 polypeptide inhibitor can be MF-438, 2-Methyl-5-(6-(4-(2-trifluoromethyl)phenoxy)piperidin-3-yl)-1,3,4-thiadiazole):

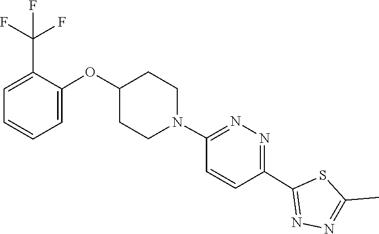 [SMILES: Cc1nnc(-c2ccc(N3CCC(Oc4ccccc4C(F)(F)F)CC3)nn2)s1]

or a pharmaceutically acceptable sale thereof. The SCD1 polypeptide inhibitor can be A939572, 4-(2-Chlorophenoxy)-N-[3-(methylcarbamoyl)phenyl]piperidine-1-carboxamide):

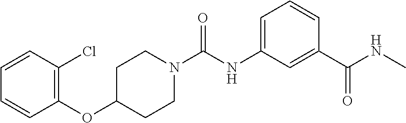 [SMILES: CNC(=O)c1cccc(NC(=O)N2CCC(Oc3ccccc3Cl)CC2)c1]

or a pharmaceutically acceptable sale thereof. The tyrosine kinase inhibitor can be cabozantinib, lenvatinib, sorafenib, sunitinib, or regorafenib. The composition can include two or more SCD1 polypeptide inhibitors. The composition can include two or more tyrosine kinase inhibitors.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1C. Cell proliferation in HCC cell lines HLE (FIG. 1A), HLF (FIG. 1B), and PLC/PRF5 (FIG. 1C) after incubation with SSI-4 alone, tyrosine kinase inhibitor (e.g., cabozantinib and lenvatinib) alone, or a combination of SSI-4 and tyrosine kinase inhibitor. The dotted line in each graph is the IC₅₀ threshold.

FIGS. 2A-2D. Tumor size of three-dimensional HCC spheroids treated with SSI-4 alone, tyrosine kinase inhibitor (e.g., cabozantinib and lenvatinib) alone, or a combination of SSI-4 and tyrosine kinase inhibitor. Two HCC PDX models LIV-58 and PAX-148 were subcutaneously implanted into NSG. SSI-4, lenvatinib and cabozantinib was given orally daily at 10 mg/kg, 2.5 mg/kg and 15 mg/kg respectively. The tumor volume was measured and recorded twice weekly from the initial treatment to tumor harvest.

FIGS. 3A-3B. SSI-4 and tyrosine kinase inhibitors in hepatocellular carcinoma cell lines. HLE (FIG. 3A) and HLF (FIG. 3B) HCC cell lines were plated in triplicate at 3×10⁴ cells/well in 1.25% FBS DMEM media and treated with SSI-4 (0.1-20,000 nM), cabozantinib (500-400,000 nM), or lenvatinib (100-320,000 nM). After a total incubation of 96 hours, media was aspirated; cells were washed with PBS and frozen at −80° C. for at least 1 hour to allow for cell lysis. CyQUANT, a fluorometric assay, determined cellular quantification, measured at an excitation of 485 nm and an emission wavelength of 538 nm. Growth inhibition was calculated as a fraction of the control (DMSO only). The dotted line indicates half-maximal inhibitory concentration (IC₅₀).

FIGS. 4A-4B. SCD1 inhibitor candidates in hepatocellular carcinoma cell lines. HLE (FIG. 4A) and HLF (FIG. 4B) HCC cell lines were plated in triplicate at 3×10⁴ cells/well in 1.25% FBS DMEM media and treated with SSI-2, MF-438 and A939572 (0.1-20,000 nM). After a total incubation of 96 hours, media was aspirated; cells were washed with PBS and frozen at −80° C. for at least 1 hour to allow for cell lysis. CyQUANT, a fluorometric assay, determined cellular quantification, measured at an excitation of 485 nm and an emission wavelength of 538 nm. Growth inhibition was calculated as a fraction of the control (DMSO only). The dotted line indicates half-maximal inhibitory concentration (IC₅₀).

FIGS. 5A-5B. Synergistic effects of SSI-4 in combination with tyrosine kinase inhibitors in hepatocellular carcinoma cell lines. Dose-dependent effect of SSI-4 alone and in combination with cabozantinib (500-400,000 nM) or Lenvatinib (100-320,000 nM) in HLE (FIG. 5A) and HLF (FIG. 5B) cells. Cells were plated in triplicate at 3×10⁴ cells/well (1.25% FBS media). After a total incubation of 96 hours, media was aspirated; cells were washed with PBS and frozen at −80° C. for at least 1 hour to allow for cell lysis. CyQUANT, a fluorometric assay, determined cellular quantification, measured at an excitation of 485 nm and an emission wavelength of 538 nm. Growth inhibition was calculated as a fraction of the control (DMSO only). The dotted line indicates half-maximal inhibitory concentration (IC₅₀). Growth inhibition and fraction of HLE or HLF cells affected (Fa) by SSI-4 combinations with cabozantinib or lenvatinib were analyzed by Chou-Talalay median-effects model (COMPUSYN) to calculate confidence index scores (CI<1.0 indicates synergy).

FIGS. 6A-6B. Synergistic effects of SSI-2 in combination with tyrosine kinase inhibitors in hepatocellular carcinoma cell lines. Dose-dependent effect of SSI-2 alone and in combination with cabozantinib (500-400,000 nM) or Lenvatinib (100-320,000 nM) in HLE (FIG. 6A) and HLF (FIG. 6B) cells. Cells were plated in triplicate at 3×10⁴ cells/well (1.25% FBS media). After a total incubation of 96 hours, media was aspirated; cells were washed with PBS and frozen at −80° C. for at least 1 hour to allow for cell lysis. CyQUANT, a fluorometric assay, determined cellular quantification, measured at an excitation of 485 nm and an emission wavelength of 538 nm. Growth inhibition was calculated as a fraction of the control (DMSO only). The dotted line indicates half-maximal inhibitory concentration (IC₅₀). Growth inhibition and fraction of HLE or HLF cells affected (Fa) by SSI-2 combinations with cabozantinib or lenvatinib were analyzed by Chou-Talalay median-effects model (COMPUSYN) to calculate confidence index scores (CI<1.0 indicates synergy).

FIGS. 7A-7B. Synergistic effects of A939572 in combination with tyrosine kinase inhibitors in hepatocellular carcinoma cell lines. Dose-dependent effect of A939572 alone and in combination with cabozantinib (500-400,000 nM) or Lenvatinib (100-320,000 nM) in HLE (FIG. 7A) and HLF (FIG. 7B) cells. Cells were plated in triplicate at 3×10⁴ cells/well (1.25% FBS media). After a total incubation of 96 hours, media was aspirated; cells were washed with PBS and frozen at −80° C. for at least 1 hour to allow for cell lysis. CyQUANT, a fluorometric assay, determined cellular quantification, measured at an excitation of 485 nm and an emission wavelength of 538 nm. Growth inhibition was calculated as a fraction of the control (DMSO only). The dotted line indicates half-maximal inhibitory concentration (IC₅₀). Growth inhibition and fraction of HLE or HLF cells affected (Fa) by A939572 combinations with cabozantinib or lenvatinib were analyzed by Chou-Talalay median-effects model (COMPUSYN) to calculate confidence index scores (CI<1.0 indicates synergy).

FIGS. 8A-8B. Synergistic effects of MF-438 in combination with tyrosine kinase inhibitors in hepatocellular carcinoma cell lines. Dose-dependent effect of MF-438 alone and in combination with cabozantinib (500-400,000 nM) or Lenvatinib (100-320,000 nM) in HLE (FIG. 8A) and HLF (FIG. 8B) cells. Cells were plated in triplicate at 3×10⁴ cells/well (1.25% FBS media). After a total incubation of 96 hours, media was aspirated; cells were washed with PBS and frozen at −80° C. for at least 1 hour to allow for cell lysis. CyQUANT, a fluorometric assay, determined cellular quantification, measured at an excitation of 485 nm and an emission wavelength of 538 nm. Growth inhibition was calculated as a fraction of the control (DMSO only). The dotted line indicates half-maximal inhibitory concentration (IC₅₀). Growth inhibition and fraction of HLE or HLF cells affected (Fa) by MF-438 combinations with cabozantinib or lenvatinib were analyzed by Chou-Talalay median-effects model (COMPUSYN) to calculate confidence index scores (CI<1.0 indicates synergy).

### DETAILED DESCRIPTION

This document provides methods and materials for treating cancer. For example, this document provides methods and materials for using one or more (e.g., one, two, three, four, or more) SCD1 polypeptide inhibitors (e.g., a SSI such as SSI-4) in combination with one or more (e.g., one, two, three, four, or more) tyrosine kinase inhibitors (e.g., cabozantinib and/or lenvatinib) to treat cancers (e.g., cancers that exhibit little or no response to treatment with either inhibitor alone). In some cases, a mammal (e.g., a human) having cancer (e.g., liver cancer such as HCC) can be administered, or can be instructed to self-administer, any one or more (e.g., one, two, three, four, or more) SCD1 polypeptide inhibitors (e.g., a SSI such as SSI-4) and any one or more (e.g., one, two, three, four, or more) tyrosine kinase inhibitors (e.g., cabozantinib and/or lenvatinib).

In some cases, one or more SCD1 polypeptide inhibitors (e.g., a SSI such as SSI-4) and one or more tyrosine kinase inhibitors (e.g., cabozantinib and/or lenvatinib) can be administered to a mammal (e.g., a human) in need thereof (e.g., a human having cancer such as HCC) to reduce the size of the cancer in the mammal. For example, one or more SCD1 polypeptide inhibitors (e.g., a SSI such as SSI-4) and one or more tyrosine kinase inhibitors (e.g., cabozantinib and/or lenvatinib) can be administered to a mammal (e.g., a human) in need thereof (e.g., a human having cancer such as HCC) as described herein to reduce the size of the cancer in the mammal. In some cases, the methods and materials provided herein can be used as described herein to reduce the number of cancer cells in the mammal by, for example, 30, 40, 50, 60, 70, 80, 90, 95, or more percent. In some cases, the methods and materials provided herein can be used as described herein to reduce the volume of one or more tumors in the mammal by, for example, 30, 40, 50, 60, 70, 80, 90, 95, or more percent.

In some cases, one or more SCD1 polypeptide inhibitors (e.g., a SSI such as SSI-4) and one or more tyrosine kinase inhibitors (e.g., cabozantinib and/or lenvatinib) can be administered to a mammal (e.g., a human) in need thereof (e.g., a human having cancer such as HCC) to improve survival of the mammal. For example, one or more SCD1 polypeptide inhibitors (e.g., a SSI such as SSI-4) and one or more tyrosine kinase inhibitors (e.g., cabozantinib and/or lenvatinib) can be administered to a mammal (e.g., a human) in need thereof (e.g., a human having cancer such as HCC) as described herein to improve survival of the mammal. In some cases, the methods and materials provided herein can be used as described herein to improve the survival of the mammal by, for example, 30, 40, 50, 60, 70, 80, 90, 95, or more percent. In some cases, the methods and materials provided herein can be used as described herein to improve the survival of the mammal by, for example, at least 6 months (e.g., about 6 months, about 8 months, about 10 months, about 1 year, about 1.5 years, about 2 years, about 2.5 years, about 3 years, about 4 years, about 5 years, or more).

Any appropriate mammal having cancer (e.g., liver cancer such as HCC) can be treated as described herein (e.g., by administering one or more SCD1 polypeptide inhibitors (e.g., a SSI such as SSI-4) and one or more tyrosine kinase inhibitors (e.g., cabozantinib and/or lenvatinib)). Examples of mammals that can have cancer and can be treated as described herein include, without limitation, humans, non-human primates (e.g., monkeys), horses, bovine species, porcine species, dogs, cats, mice, and rats. In some cases, a human having cancer (e.g., liver cancer such as HCC) can be treated by administering one or more SCD1 polypeptide inhibitors (e.g., a SSI such as SSI-4) and one or more tyrosine kinase inhibitors (e.g., cabozantinib and/or lenvatinib).

A mammal (e.g., a human) having any type of cancer can be treated as described herein (e.g., by administering one or more SCD1 polypeptide inhibitors (e.g., a SSI such as SSI-4) and one or more tyrosine kinase inhibitors (e.g., cabozantinib and/or lenvatinib)). In some cases, a cancer that can be treated as described herein can include one or more solid tumors. In some cases, a cancer that can be treated as described herein can be a blood cancer. In some cases, a cancer treated as described herein can be resistant to one or more tyrosine kinase inhibitors (e.g., cabozantinib and/or lenvatinib). Examples of cancers that can be treated as described herein include, without limitation, liver cancers (e.g., HCC), thyroid cancers (e.g., differentiated thyroid cancer (DTC)), renal cell carcinomas (e.g., advanced renal cell carcinoma (RCC)), and endometrial carcinomas.

In some cases, the methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and genetic tests. Once identified as having cancer, a mammal can be treated as described herein (e.g., by administering one or more SCD1 polypeptide inhibitors (e.g., a SSI such as SSI-4) and one or more tyrosine kinase inhibitors (e.g., cabozantinib and/or lenvatinib)).

A mammal (e.g., a human) having cancer (e.g., liver cancer such as HCC) can be administered, or can be instructed to self-administer, any appropriate one or more (e.g., one, two, three, four, or more) SCD1 polypeptide inhibitors. A SCD1 polypeptide inhibitor can inhibit SCD1 polypeptide activity or can inhibit SCD1 polypeptide expression. In some cases, a SCD1 polypeptide inhibitor can inhibit one or more molecules upstream of a SCD1 polypeptide in the monounsaturated fatty acid (MUFA) pathway (e.g., a fatty acid synthase (FASN) polypeptide and/or an acetyl-CoA carboxylase (ACC) polypeptide). Examples of compounds that can inhibit SCD1 polypeptide activity include, without limitation, antibodies (e.g., neutralizing antibodies) that target (e.g., target and bind) to a SCD1 polypeptide, small molecules that target (e.g., target and bind) to a SCD1 polypeptide, antibodies (e.g., neutralizing antibodies) that target (e.g., target and bind) to a FASN polypeptide, small molecules that target (e.g., target and bind) to a FASN polypeptide, antibodies (e.g., neutralizing antibodies) that target (e.g., target and bind) to an ACC polypeptide, and small molecules that target (e.g., target and bind) to an ACC polypeptide. Examples of compounds that can inhibit of SCD1 polypeptide expression include, without limitation, nucleic acid molecules designed to induce RNA interference of polypeptide expression of a SCD1 polypeptide (e.g., a siRNA molecule or a shRNA molecule), antisense molecules, miRNAs, and CRISPR RNAs.

In some cases, a SCD1 polypeptide inhibitor can have Formula (I):

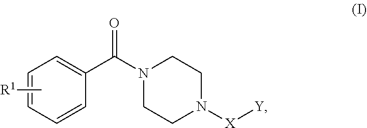

or a pharmaceutically acceptable salt thereof, where R¹ is an unsubstituted C₁₋₆ alkyl or C₁₋₆ haloalkyl; X is

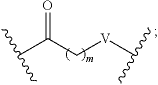

Y is selected from:

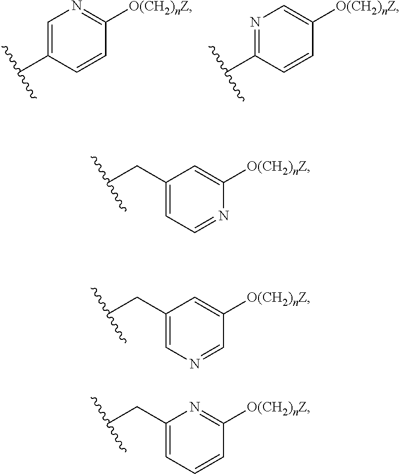

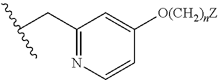

and

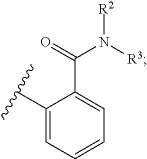

m is 0 or 1; n is 0, 1, or 2; V is NR⁴ or O; R², R³, and R⁴ are each independently H or an unsubstituted C₁₋₆ alkyl; and Z is an unsubstituted aryl. In some cases, a SCD1 polypeptide inhibitor according to Formula (I) can have the structure of Formula (Ia):

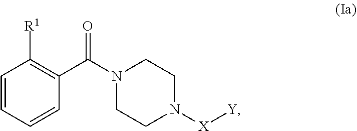

or a pharmaceutically acceptable salt thereof. Representative examples of SCD1 polypeptide inhibitors according to Formula (I) and/or Formula (Ia) include, without limitation:

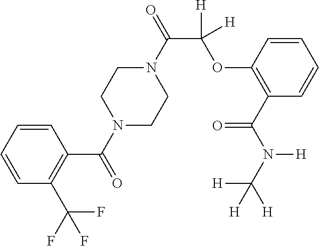 [SMILES: CNC(=O)c1ccccc1OCC(=O)N1CCN(C(=O)c2ccccc2C(F)(F)F)CC1]

SSI-1 (N-Methyl-2-(2-oxo-2-{4-[2-(trifluoromethyl)benzoyl]piperazin-1-yl}ethoxy)benzamide) or a pharmaceutically acceptable salt thereof;

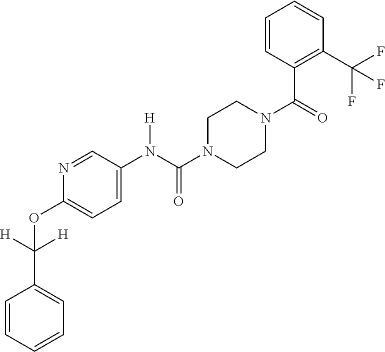 [SMILES: O=C(Nc1ccc(OCc2ccccc2)nc1)N1CCN(C(=O)c2ccccc2C(F)(F)F)CC1]

SSI-2 (2-(benzyloxy)-5-{[hydroxy({4-[2-(trifluoromethyl)benzoyl]piperazin-1-yl})methyl]amino}-1,2-dihydropyridin-2-ylium-1-ide) or a pharmaceutically acceptable salt thereof; and

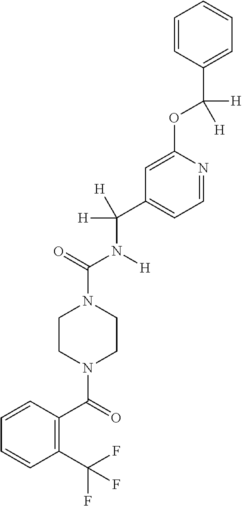 [SMILES: O=C(NCc1ccnc(OCc2ccccc2)c1)N1CCN(C(=O)c2ccccc2C(F)(F)F)CC1]

SSI-3 (2-(benzyloxy)-4-({[hydroxy({4-[2-(trifluoromethyl)benzoyl]piperazin-1-yl})methyl]azanidyl}methyl)-1,2-dihydropyridin-2-ylium-1-ide) or a pharmaceutically acceptable salt thereof.

In some cases, a SCD1 polypeptide inhibitor can have Formula (II):

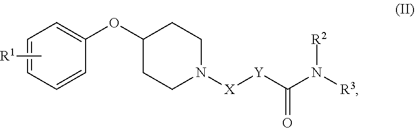

or pharmaceutically acceptable salt thereof, where R¹ is halo; X is —(C═O)NR⁴—; Y is

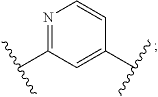 [SMILES: *c1ccnc(*)c1]

R², R³, and R⁴ are each independently H or an unsubstituted C₁₋₆ alkyl. In some cases, a SCD1 polypeptide inhibitor according to Formula (II) can have the structure of Formula (IIa):

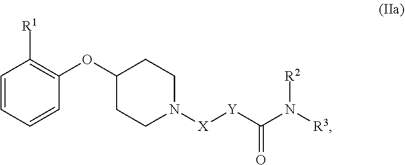

or pharmaceutically acceptable salt thereof. A representative example of a SCD1 polypeptide inhibitor according to Formula (II) and/or Formula (IIa) include:

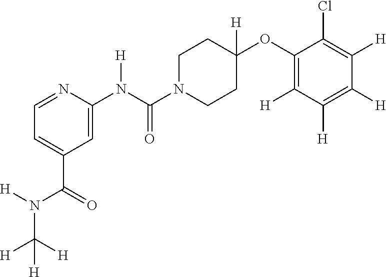 [SMILES: CNC(=O)c1ccnc(NC(=O)N2CCC(Oc3ccccc3Cl)CC2)c1]

SSI-4 (2-{[4-(2-Chlorophenoxy)piperidine-1-carbonyl]amino}-N-methylpyridine-4-carboxamide) or pharmaceutically acceptable salt thereof.

In some cases, a SCD1 polypeptide inhibitor can have the structure:

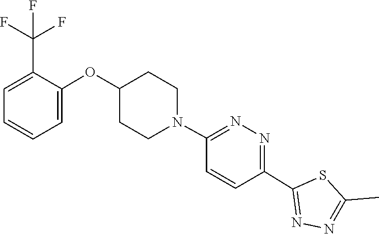 [SMILES: Cc1nnc(-c2ccc(N3CCC(Oc4ccccc4C(F)(F)F)CC3)nn2)s1]

MF-438 (2-Methyl-5-(6-(4-(2-trifluoromethyl)phenoxy)piperidin-3-yl)-1,3,4-thiadiazole) or a pharmaceutically acceptable sale thereof.

In some cases, a SCD1 polypeptide inhibitor can have the structure:

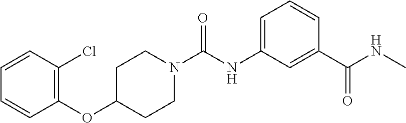 [SMILES: CNC(=O)c1cccc(NC(=O)N2CCC(Oc3ccccc3Cl)CC2)c1]

A939572 (4-(2-Chlorophenoxy)-N-[3-(methylcarbamoyl)phenyl]piperidine-1-carboxamide) or a pharmaceutically acceptable sale thereof.

In some cases, a SCD1 polypeptide inhibitor can be as described elsewhere (see, e.g., WO 2016/022955 at, for example, pages 13-17).

A mammal (e.g., a human) having cancer (e.g., liver cancer such as HCC) can be administered, or can be instructed to self-administer, any appropriate one or more (e.g., one, two, three, four, or more) tyrosine kinase inhibitors. A tyrosine kinase inhibitor can inhibit any appropriate tyrosine kinase. Examples of tyrosine kinases that can be inhibited by a tyrosine kinase inhibitor as described herein include, without limitation, vascular endothelial growth factor receptors (VEGFRs; e.g., VEGFR1, VEGFR2, and VEGFR3), platelet-derived growth factor receptors (PDGFRs; e.g., PDGFRa), fibroblast growth factor receptors (FGFRs; e.g., FGFR1, FGFR2, FGFR3, and FGFR4), a receptor tyrosine kinase encoded by a c-KIT proto-oncogene, a receptor tyrosine kinase encoded by a RET proto-oncogene, hepatocyte growth factor receptors (HGFRs; also referred to as c-MET), a receptor tyrosine kinase encoded by an AXL proto-oncogene, FMS like tyrosine kinase 3 (FLT-3), tropomyosin receptor kinase B (TRKB), and TIE-2.

A receptor tyrosine kinase inhibitor can inhibit receptor tyrosine kinase polypeptide activity or can inhibit receptor tyrosine kinase polypeptide expression. Examples of compounds that can inhibit receptor tyrosine kinase polypeptide activity include, without limitation, antibodies (e.g., neutralizing antibodies) that target (e.g., target and bind) to a receptor tyrosine kinase polypeptide, and small molecules that target (e.g., target and bind) to a receptor tyrosine kinase polypeptide. Examples of compounds that can inhibit of receptor tyrosine kinase polypeptide expression include, without limitation, nucleic acid molecules designed to induce RNA interference of polypeptide expression of a receptor tyrosine kinase polypeptide (e.g., a siRNA molecule or a shRNA molecule), antisense molecules, miRNAs, and CRISPR RNAs. Examples of receptor tyrosine kinase inhibitors that can be administered to mammal (e.g., a human) having cancer (e.g., liver cancer such as HCC) as described herein include, without limitation, cabozantinib, lenvatinib, sorafenib, sunitinib, and regorafenib.

In some cases, one or more SCD1 polypeptide inhibitors (e.g., a SSI such as SSI-4) and/or one or more tyrosine kinase inhibitors (e.g., cabozantinib and/or lenvatinib) can be formulated into a composition (e.g., a pharmaceutically acceptable composition) for administration to a mammal (e.g., a human) having cancer (e.g., liver cancer such as HCC). For example, one or more SCD1 polypeptide inhibitors (e.g., a SSI such as SSI-4) and one or more tyrosine kinase inhibitors (e.g., cabozantinib and/or lenvatinib) can be formulated into a single composition. In some cases, one or more SCD1 polypeptide inhibitors (e.g., a SSI such as SSI-4) and/or one or more tyrosine kinase inhibitors (e.g., cabozantinib and/or lenvatinib) can be formulated together with one or more pharmaceutically acceptable carriers (additives), excipients, and/or diluents. In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein (e.g., a pharmaceutically acceptable composition including one or more SCD1 polypeptide inhibitors (e.g., a SSI such as SSI-4) and/or one or more tyrosine kinase inhibitors (e.g., cabozantinib and/or lenvatinib)) include, without limitation, serum proteins (e.g., human serum albumin), water, and salts or electrolytes (e.g., phosphate salts, saline, protamine sulfate, and DMSO).

In some cases, the one or more SCD1 polypeptide inhibitors (e.g., a SSI such as SSI-4) and the one or more tyrosine kinase inhibitors (e.g., cabozantinib and/or lenvatinib) can be administered to the mammal at the same time. For example, one or more SCD1 polypeptide inhibitors (e.g., a SSI such as SSI-4) and one or more tyrosine kinase inhibitors (e.g., cabozantinib and/or lenvatinib) can be administered to a mammal (e.g., a human) having cancer (e.g., liver cancer such as HCC) in a single composition containing both the one or more SCD1 polypeptide inhibitors (e.g., a SSI such as SSI-4) and the one or more tyrosine kinase inhibitors (e.g., cabozantinib and/or lenvatinib).

In some cases, the one or more SCD1 polypeptide inhibitors (e.g., a SSI such as SSI-4) and the one or more tyrosine kinase inhibitors (e.g., cabozantinib and/or lenvatinib) can be administered to the mammal separately.

One or more SCD1 polypeptide inhibitors (e.g., a SSI such as SSI-4) and one or more tyrosine kinase inhibitors (e.g., cabozantinib and/or lenvatinib) can be administered to a mammal (e.g., a human) by any appropriate route (e.g., oral, intranasal, inhalation, transdermal, and parenteral). One or more SCD1 polypeptide inhibitors (e.g., a SSI such as SSI-4) and one or more tyrosine kinase inhibitors (e.g., cabozantinib and/or lenvatinib) can be administered to a mammal locally or systemically. For example, one or more SCD1 polypeptide inhibitors (e.g., a SSI such as SSI-4) and/or one or more tyrosine kinase inhibitors (e.g., cabozantinib and/or lenvatinib) can be administered systemically by oral administration to a mammal (e.g., a human). When a mammal (e.g., a human) is administered one or more SCD1 polypeptide inhibitors (e.g., a SSI such as SSI-4) and one or more tyrosine kinase inhibitors (e.g., cabozantinib and/or lenvatinib), and the one or more SCD1 polypeptide inhibitors and the one or more tyrosine kinase inhibitors are administered separately, the one or more SCD1 polypeptide inhibitors and the one or more tyrosine kinase inhibitors can be administered by the same route or by different routes.

One or more SCD1 polypeptide inhibitors (e.g., a SSI such as SSI-4) can be administered to a mammal (e.g., a human) having cancer (e.g., liver cancer such as HCC) in any appropriate amount (e.g., any appropriate dose). An effective amount of one or more SCD1 polypeptide inhibitors (e.g., a SSI such as SSI-4) can be any amount that can treat a mammal having cancer (e.g., HCC) without producing significant toxicity to the mammal.

One or more tyrosine kinase inhibitors (e.g., cabozantinib and/or lenvatinib) can be administered to a mammal (e.g., a human) having cancer (e.g., liver cancer such as HCC) in any appropriate amount (e.g., any appropriate dose). An effective amount of one or more tyrosine kinase inhibitors (e.g., cabozantinib and/or lenvatinib) can be any amount that can treat a mammal having cancer (e.g., HCC) without producing significant toxicity to the mammal.

The effective amount of one or more SCD1 polypeptide inhibitors (e.g., a SSI such as SSI-4) and of one or more tyrosine kinase inhibitors (e.g., cabozantinib and/or lenvatinib) can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer (e.g., liver cancer such as HCC) in the mammal being treated may require an increase or decrease in the actual effective amount administered.

One or more SCD1 polypeptide inhibitors (e.g., a SSI such as SSI-4) and one or more tyrosine kinase inhibitors (e.g., cabozantinib and/or lenvatinib) can be administered to a mammal (e.g., a human) having cancer (e.g., liver cancer such as HCC) at any appropriate frequency. The frequency of administration can be any frequency that can treat a mammal having cancer (e.g., HCC) without producing significant toxicity to the mammal. For example, the frequency of administration can be from about twice a day to about once a day. When a mammal (e.g., a human) is administered one or more SCD1 polypeptide inhibitors (e.g., a SSI such as SSI-4) and one or more tyrosine kinase inhibitors (e.g., cabozantinib and/or lenvatinib) separately, the one or more SCD1 polypeptide inhibitors and the one or more tyrosine kinase inhibitors can be administered at the same frequency or at different frequencies. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

One or more SCD1 polypeptide inhibitors (e.g., a SSI such as SSI-4) and one or more tyrosine kinase inhibitors (e.g., cabozantinib and/or lenvatinib) can be administered to a mammal (e.g., a human) having cancer (e.g., liver cancer such as HCC) for any appropriate duration. An effective duration for administering or using one or more SCD1 polypeptide inhibitors (e.g., a SSI such as SSI-4) and one or more tyrosine kinase inhibitors (e.g., cabozantinib and/or lenvatinib) can be any duration that can treat a mammal having cancer (e.g., HCC) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) as described herein (e.g., by administering one or more SCD1 polypeptide inhibitors (e.g., a SSI such as SSI-4) and one or more tyrosine kinase inhibitors (e.g., cabozantinib and/or lenvatinib)) can include administering to the mammal the one or more SCD1 polypeptide inhibitors (e.g., a SSI such as SSI-4) and the one or more tyrosine kinase inhibitors (e.g., cabozantinib and/or lenvatinib) as the sole active ingredients. For example, a composition containing one or more SCD1 polypeptide inhibitors (e.g., a SSI such as SSI-4) and one or more tyrosine kinase inhibitors (e.g., cabozantinib and/or lenvatinib) can include the one or more SCD1 polypeptide inhibitors and the one or more tyrosine kinase inhibitors as the sole active ingredients in the composition for treating a mammal (e.g., a human) having cancer (e.g., liver cancer such as HCC).

In some cases, methods for treating a mammal (e.g., a human) as described herein (e.g., by administering one or more SCD1 polypeptide inhibitors (e.g., a SSI such as SSI-4) and one or more tyrosine kinase inhibitors (e.g., cabozantinib and/or lenvatinib)) also can include administering to the mammal one or more (e.g., one, two, three, or more) additional agents used to treat cancer and/or performing one or more (e.g., one, two, three, or more) therapies used to treat cancer. For example, a combination therapy used to treat a mammal (e.g., a human) having cancer (e.g., liver cancer such as HCC) can include administering to the mammal one or more SCD1 polypeptide inhibitors (e.g., a SSI such as SSI-4) and one or more tyrosine kinase inhibitors (e.g., cabozantinib and/or lenvatinib), and administering to the mammal one or more (e.g., one, two, three, or more) additional agents used to treat cancer. In some cases, an additional agent that can be administered to a mammal to treat cancer (e.g., HCC) can be a chemotherapeutic agent. In some cases, an agent that can be administered to a mammal to treat cancer (e.g., HCC) can be an immune checkpoint inhibitor (e.g., a PD1 inhibitor, a PD-L1 inhibitor, a CTLA4 inhibitor, or combinations thereof). In some cases, an additional agent that can be administered to a mammal to treat cancer (e.g., HCC) can be a cytotoxic agent. Examples of additional agents that can be administered to a mammal (e.g., a human) having cancer (e.g., liver cancer such as HCC) to treat the mammal include, without limitation, durvalumab, nivolumab, pembrolizumab, ramucirumab, and any combinations thereof. In cases where one or more SCD1 polypeptide inhibitors (e.g., a SSI such as SSI-4) and one or more tyrosine kinase inhibitors (e.g., cabozantinib and/or lenvatinib) are used in combination with additional agents used to treat a mammal (e.g., a human) having cancer (e.g., liver cancer such as HCC), the one or more additional agents can be administered at the same time (e.g., in a single composition containing one or more SCD1 polypeptide inhibitors (e.g., a SSI such as SSI-4) and one or more tyrosine kinase inhibitors (e.g., cabozantinib and/or lenvatinib), and containing the one or more additional agents) or independently. For example, a composition including one or more SCD1 polypeptide inhibitors (e.g., a SSI such as SSI-4) and one or more tyrosine kinase inhibitors (e.g., cabozantinib and/or lenvatinib) can be administered first, and the one or more additional agents administered second, or vice versa.

In some cases, a combination therapy used to treat a mammal (e.g., a human) having cancer (e.g., liver cancer such as HCC) can include administering to the mammal one or more SCD1 polypeptide inhibitors (e.g., a SSI such as SSI-4) and one or more tyrosine kinase inhibitors (e.g., cabozantinib and/or lenvatinib), and can include performing one or more (e.g., one, two, three, or more) therapies used to treat cancer. Examples of additional therapies that can be used to treat a mammal (e.g., a human) having cancer (e.g., liver cancer such as HCC) include, without limitation, radiation therapies, and/or adoptive cell transfer therapies. In cases where one or more SCD1 polypeptide inhibitors (e.g., a SSI such as SSI-4) and one or more tyrosine kinase inhibitors (e.g., cabozantinib and/or lenvatinib) are used in combination with one or more therapies used to treat a mammal (e.g., a human) having cancer (e.g., liver cancer such as HCC), the one or more additional therapies can be performed at the same time or independently of the administration of the one or more SCD1 polypeptide inhibitors and the one or more tyrosine kinase inhibitors. For example, one or more SCD1 polypeptide inhibitors (e.g., a SSI such as SSI-4) and one or more tyrosine kinase inhibitors (e.g., cabozantinib and/or lenvatinib) can be administered before, during, or after the one or more additional therapies are performed.

In some cases, the size of the cancer (e.g., the number of cancer cells and/or the volume of one or more tumors) present within a mammal can be monitored. Any appropriate method can be used to determine whether or not the size of the cancer present within a mammal is reduced. For example, imaging techniques can be used to assess the size of the cancer present within a mammal (e.g., a human).

The invention will be further described in the following examples, which do not limit the scope of the invention described in the claims.

### EXAMPLES

### Example 1: SCD1 Blockade Provides an Enhanced and Durable Response to Tyrosine Kinase Inhibitors in Hepatocellular Carcinoma (HCC)

This Example describes the anti-tumor synergy of SSI-4 and one or more tyrosine kinase inhibitors (e.g., cabozantinib and/or lenvatinib) resulting in enhanced tumor regression (e.g., as compared to treating with only a single agent).

### Materials and Methods

### Human Cell Lines and Patient-Derived Xenografts (PDXs)

Human hepatocellular carcinoma (HCC) cell lines HLE, HLF, and PLC/PRF5 were purchased from ATCC (Manassas, VA). HCC cell lines were maintained in DMEM media (Gibco, Waltham, MA) supplemented with 10% fetal bovine serum (HyClone Logan, UT), 10 mM HEPES, 1 mM sodium pyruvate, 1% non-essential amino acids (all three from Corning Incorporated, Corning, NY), 1% penicillin-streptomycin-amphotericin B (Cellgro Herndon, VA). Cell lines were incubated at 37° C. with 5% CO₂ in a humidified atmosphere incubator (NuAire, Plymouth, MN). LIV-58 and PAX-148 patient-derived xenografts (PDXs) were developed from human patient HCC tissue.

### 3D Cell Culture

HCC PDX tumor tissues were isolated from mice when a tumor reached around 2000 mm³. Tumor dissociation was performed using a human dissociation kit and gentleMACS™ Dissociator (Miltenyi Biotec Gaithersburg, MD) following manufacturer protocol. Obtained single-cell suspensions were washed through a 70 μm strainer (Genesee Scientific, El Cajon, CA), and cells were incubated with red blood cell lysis (RBC) buffer (Miltenyi Biotec Gaithersburg, MD). Cells were washed and resuspended with 3D culture medium and cultured in ultra-low attachment flasks (ULA; Corning Incorporated, Corning, NY) until cancer tumor spheres were developed.

### Compounds

Tyrosine kinase inhibitors cabozantinib and lenvatinib were purchased from MedKoo Biosciences (Morrisville, NC). Cabozantinib-malate was purchased from Selleck Chemicals (Houston, TX) and dissolved in DMSO for storage at −20° C.

### Anti-Proliferative Assay In Vitro

HCC cell lines (3×10⁵ cells) were suspended in a 10 mL culture medium and seeded onto 96-well black plates at 100 μL per well (Corning Incorporated, Corning, NY, USA). The next day, SSI-4, cabozantinib, and/or lenvatinib were added in concentrations ranging from 120 μM to 0.1 nM and incubated with cells for 96 hours. After incubation times, the media was discarded, and cells were washed with 100 μL PBS, dried, and placed at −80° C. for at least 30 minutes. Next, a CyQUANT® Cell Proliferation Assay (Thermo Fisher, Waltham, MA) was used for quantifying cells and for assessing cell proliferation and cytotoxicity following manufacturer protocol. For 3D cell culture, the cell viability was analyzed using CellTiter-Glo® 3D Cell Viability Assay (Promega, Madison, WI), and relative luminescence units (RLU) were measured. Based, on the fluorescence or luminescence values, the percentage of cell proliferation inhibition and half-maximal inhibitory concentration (IC₅₀) was determined using the GraphPad Prism (Dotmatics, San Diego, CA). The resulting combination index (CI) data based on the median effect principle proposed by Chou and Talalay was generated using CalcuSyn Software Version 2.0 (Biosoft). At least 3 independent experiments were performed.

### Western Blot

Cells were washed in PBS solution (Corning Incorporated, Corning, NY, USA), then RIPA buffer (Thermo Fisher, Waltham, MA) containing proteases inhibitor (Promega, Madison, WI) and phosphatases inhibitor (Thermo Fisher, Waltham, MA) was added. Cells were incubated on ice for 20 minutes and centrifuged at 16,000×g for 10 minutes at 4° C. Supernatant was collected and protein concentration was measured using the Pierce™ BCA Protein Assay Kit (Thermo Fisher, Waltham, MA) following manufacturer protocol. Samples were diluted to 3 μg/μL in NuPAGE LDS Sample Buffer (4X) and molecular grade water (both Thermo Fisher, Waltham, MA). 25 μg protein per sample was used. Samples were heated for 5 minutes at 100° C. and then separated on 4-12% gradient polyacrylamide NuPAGE Bis-Tris protein gels (Thermo Fisher, Waltham, MA). The separated samples were then transferred to a 0.45 m PVDF membrane, and protein transfer was carried out for 7 min using iBlot™ 2 Gel Transfer Kit (Thermo Fisher, Waltham, MA). The PVDF membranes were blocked using a 5% blocking solution (Membrane Blocking Solution, Thermo Fisher, Waltham, MA) at 4° C. overnight. Next, membranes were incubated overnight at 4° C. with primary antibodies. Then membranes were incubated for 1 hour with horseradish peroxidase-conjugated anti-rabbit or anti-mouse antibodies (Jackson ImmunoResearch Inc, West Grove, PA) diluted at 1:10,000. The next step was incubation in the dark for 5 minutes with SuperSignal™ West Pico PLUS Chemiluminescent Substrate (Thermo Fisher, Waltham, MA). The chemiluminescent intensity was measured using ChemiDoc Imaging Systems (BioRad, Hercules, California). Densitometric analysis was performed using ImageJ 1.46r software (NIH, Bethesda, MA).

### Tissue Microarray (TMA) and Immunohistochemistry (IHC)

Immunostaining was performed on paraffin-embedded tissue. Thyroid tissue microarray (TMA) was made from archival samples. Paraffin blocks were cut into 5 μm thick sections. Slides were deparaffinized, rehydrated with decreasing concentrations of ethanol, and finally in the water. Antigen retrieval was performed using Antigen Retrieval Solution (pH 6 or pH 9) (Dako, Glostrup, Denmark) for at least 15 minutes in an autoclave, and cooling for 30 minutes at room temperature. The activity of endogenous peroxidases was blocked by washing slides with 3% hydrogen peroxide (Fisher Scientific, Waltham, MA) for 10 minutes. Primary antibodies were used for 60 minutes at room temperature, antibodies were used. The primary antibody was visualized using the Envision Dual Labeled Polymer kit (Dako, Glostrup, Denmark) according to the manufacturer's instructions. After washing slides were counterstained with Gill I Hematoxylin (Sigma-Aldrich, Burlington, MA). Slides were dehydrated through ethanol and xylene and cover-slipped using a xylene-based mounting medium (Fisher Scientific, Waltham, MA, USA). Samples were examined under bright-field illumination at ×20 objectives, and digital images were obtained using Aperio AT2 (Leica, Wetzlar, Germany). Results were processed using Aperio eSlide Manager and H-Score values were estimated using the Aperio ImageScope Software (both Aperio Technologies, Vista, CA, USA). Normal tissues were used as a positive or negative control.

### In Vivo Analysis

HCC PDX LIV-58 or PAX-148 tissue (around 25 mm³) was implanted subcutaneously (s.c.) into the right flank of the NSG mouse, in 100 μL of Matrigel Matrix (Corning Incorporated, Corning, NY). The administration of the compound to the mice was started when the tumors reached a size of 75-100 mm³. SSI-4 was resuspended in 10% Captisol (company), and cabozantinib and lenvatinib were prepared in 5% Tween-80, 30% PEG-400 (both Millipore, Burlington, MA), and 75% saline. Mice were fed daily using an oral gavage needle. Tumor volumes were calculated using the formula 0.5236 (L x W x H) and body weight was measured every 3 days.

### Statistical Analysis

Statistical analysis and graphs were prepared using GraphPad Prism version 6.04 for Windows (GraphPad Software, La Jolla, CA). Before choosing the test, the normality of the distribution was checked by Levene's test. Data showing normal distribution were analyzed using the t-test or one-way ANOVA test. Non-parametric data were analyzed using a Kruskal-Wallis test. Results were of statistical significance where p<0.05. The graphs were prepared using GraphPad Prism version 6.04 for Windows (GraphPad Software, La Jolla, CA, USA). Statistically significant results are indicated by an asterisk (*).

### Results

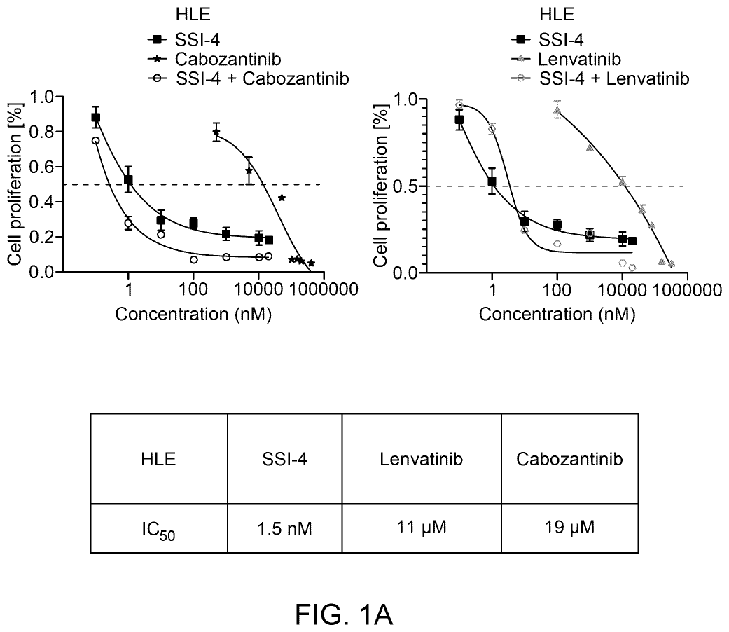

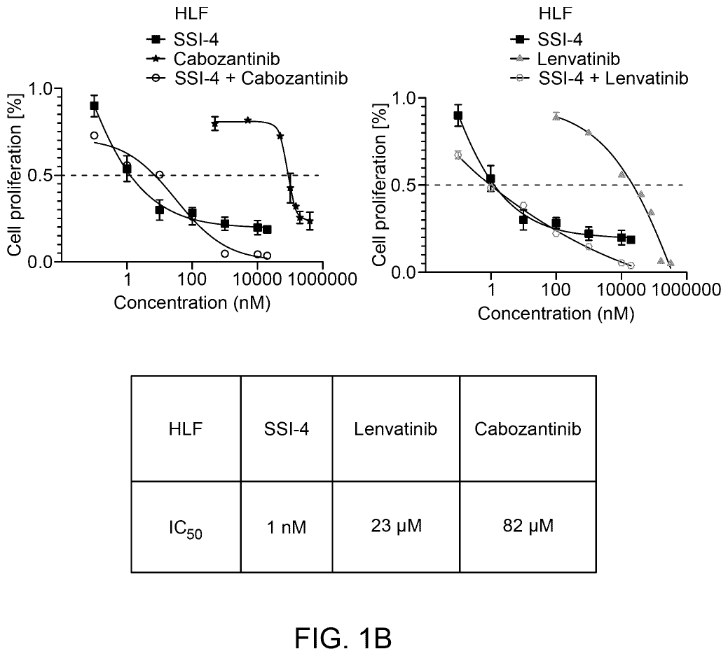

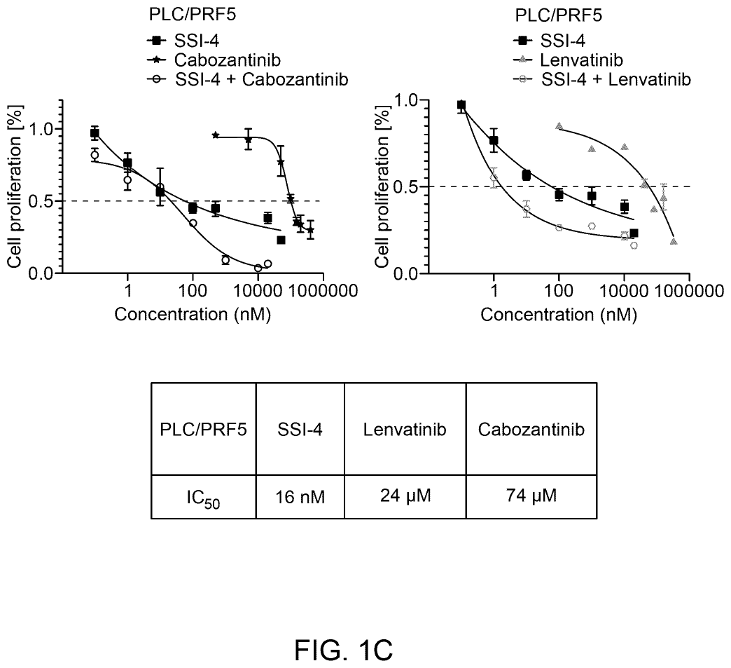

SSI-4 with cabozantinib or lenvatinib showed a synergistic effect in HCC cells Cell proliferation assay was used to determine HCC cells response to the SSI-4 alone, cabozantinib alone, SSI-4 in combination with cabozantinib, lenvatinib alone, and SSI-4 in combination with Lenvatinib. Three HCC cell lines (HLE, HLF, and PLC/PRF5) were tested and the concentration that inhibits proliferation of 50% cells (IC₅₀) was determined. The IC₅₀ for SSI-4 was 1 to 19 nM, cabozantinib from 19 to 72 μM, and for lenvatinib 11 to 24 μM (FIG. 1). The combined use of SSI-4 and cabozantinib or SSI-4 and lenvatinib showed synergistic activity against all used cell lines. Tables with combination index values for drug interactions are shown below.

TABLE 1
IC50 values for combination treatments shown in FIG. 1A.
CI For experimental values HLE

| SSI-4 | Cabozantinib | | | |
|---|---|---|---|---|
| (nM) | (nM) | Fa | CI | Effect |
| 0.1 | 500 | 0.249644 | 2.232 | antagonism |
| 1 | 5000 | 0.72085 | 0.338 | synergism |
| 10 | 50000 | 0.788942 | 1.833 | antagonism |
| 100 | 10000 | 0.93018 | 0.046 | synergism |
| 1000 | 150000 | 0.91465 | 0.979 | synergism |
| 10000 | 200000 | 0.915224 | 0.345 | synergism |
| 20000 | 400000 | 0.910128 | 0.312 | synergism |

CI For experimental values HLE

| SSI-4 | Lenvatinib | | | |
|---|---|---|---|---|
| (nM) | (nM) | Fa | CI | Effect |
| 0.1 | 100 | 0.033135 | 1.63E+04 | antagonism |
| 1 | 1000 | 0.171417 | 92.641 | antagonism |
| 10 | 10000 | 0.757103 | 0.245 | synergism |
| 100 | 40000 | 0.833675 | 0.467 | synergism |
| 1000 | 80000 | 0.774806 | 2.355 | antagonism |
| 10000 | 160000 | 0.945069 | 0.283 | synergism |
| 20000 | 320000 | 0.97287 | 0.179 | synergism |

TABLE 2
IC50 values for combination treatments shown in FIG. 1B.
CI For experimental values HLF

| SSI-4 | Cabozantinib | | | |
|---|---|---|---|---|
| (nM) | (nM) | Fa | CI | Effect |
| 0.1 | 500 | 0.270341 | 0.716 | synergism |
| 1 | 5000 | 0.443991 | 1.499 | antagonism |
| 10 | 50000 | 0.496104 | 2.556 | antagonism |
| 100 | 10000 | 0.737476 | 0.176 | synergism |
| 1000 | 150000 | 0.952866 | 0.003 | synergism |
| 10000 | 200000 | 0.95442 | 0.008 | synergism |
| 20000 | 400000 | 0.964315 | 0.006 | synergism |

CI For experimental values HLF

| SSI-4 | Lenvatinib | | | |
|---|---|---|---|---|
| (nM) | (nM) | Fa | CI | Effect |
| 0.1 | 100 | 0.328209 | 0.218 | synergism |
| 1 | 1000 | 0.519342 | 0.177 | synergism |
| 10 | 10000 | 0.616516 | 1.684 | antagonism |
| 100 | 40000 | 0.776991 | 0.652 | synergism |
| 1000 | 80000 | 0.855118 | 0.563 | synergism |
| 10000 | 160000 | 0.945656 | 0.168 | synergism |
| 20000 | 320000 | 0.96161 | 0.176 | synergism |

TABLE 3
IC50 values for combination treatments shown in FIG. 1C.
CI For experimental values PLC/PRF5

| SSI-4 | Cabozantinib | | | |
|---|---|---|---|---|
| (nM) | (nM) | Fa | CI | Effect |
| 0.1 | 500 | 0.178284 | 0.12 | synergism |
| 1 | 5000 | 0.351347 | 0.176 | synergism |
| 10 | 50000 | 0.400001 | 1.195 | antagonism |
| 100 | 10000 | 0.649877 | 0.08 | synergism |
| 1000 | 150000 | 0.906815 | 0.04 | synergism |
| 10000 | 200000 | 0.961844 | 0.011 | synergism |
| 20000 | 400000 | 0.93216 | 0.068 | synergism |

CI For experimental values PLC/PRF5

| SSI-4 | Lenvatinib | | | |
|---|---|---|---|---|
| (nM) | (nM) | Fa | CI | Effect |
| 0.1 | 100 | 0.010312 | 1280.973 | antagonism |
| 1 | 1000 | 0.44817 | 0.175 | synergism |
| 10 | 10000 | 0.629834 | 0.494 | synergism |
| 100 | 40000 | 0.737965 | 0.849 | synergism |
| 1000 | 80000 | 0.727849 | 0.951 | synergism |
| 10000 | 160000 | 0.783691 | 0.641 | synergism |
| 20000 | 320000 | 0.840717 | 2.593 | antagonism |

SSI-4 with Cabozantinib or Lenvatinib Showed a Synergistic Effect in HCC PDX Models

3-D spheroids were developed from LIV-58 PDX model, and drug were used to recapitulated patient's response to therapy in vivo.

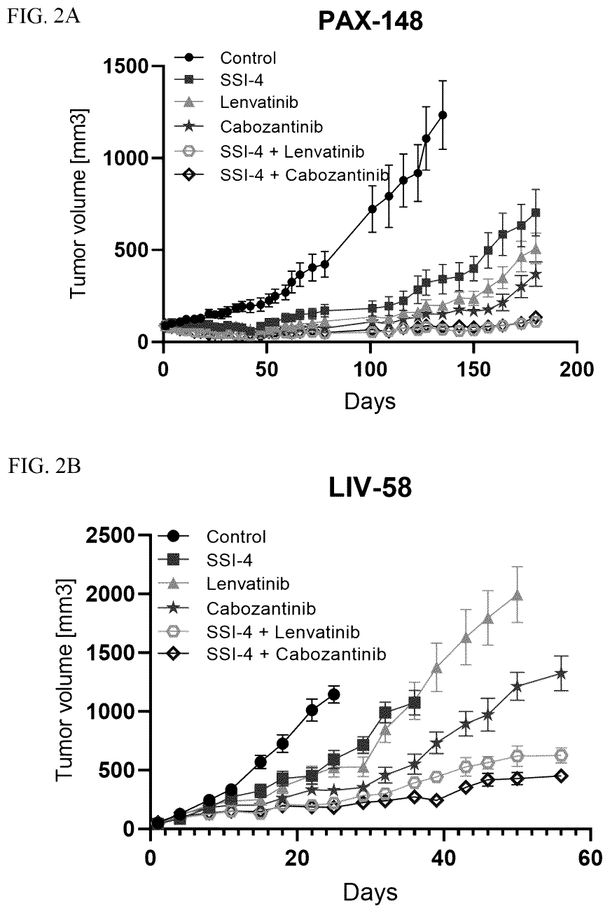

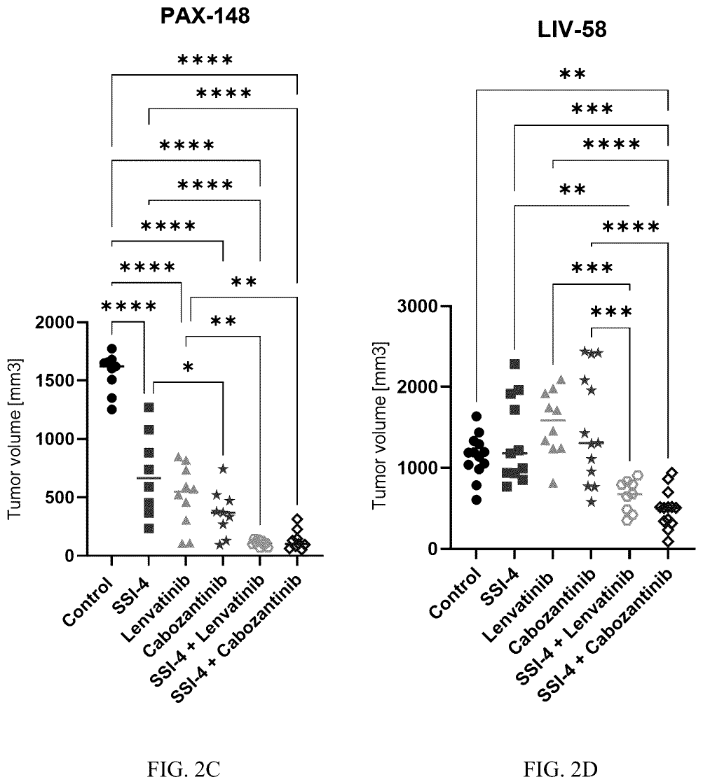

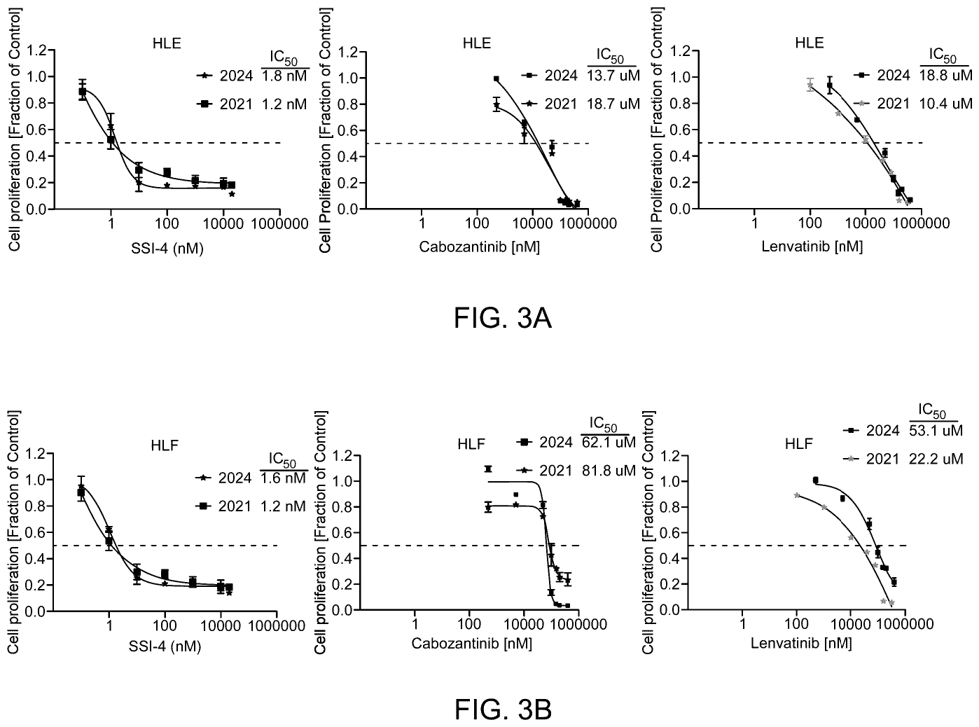

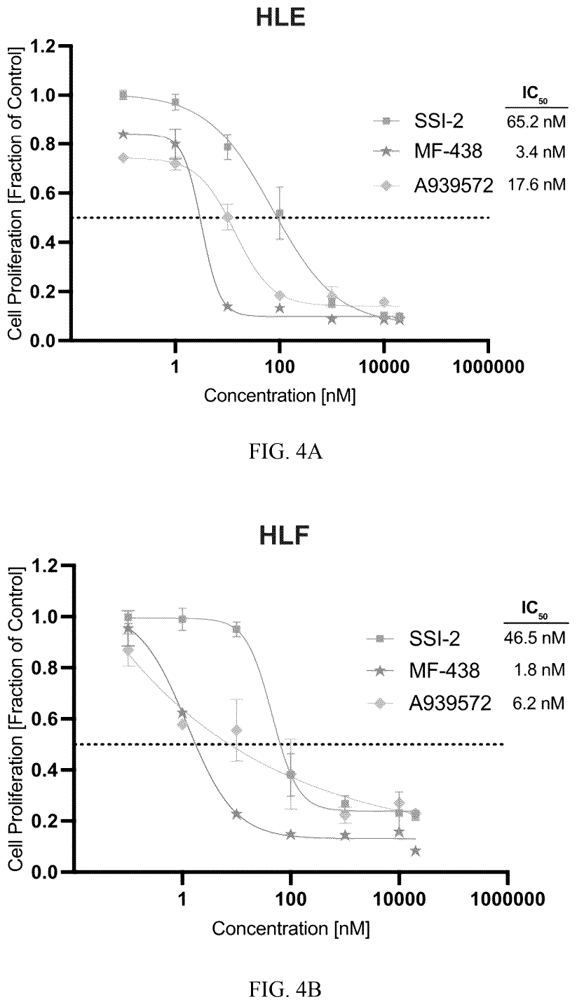

Combination therapy using SSI-4 with cabozantinib and lenvatinib was used in two HCC PDX models PAX-148 and LIV-58. Results showed that after time both models start to become resistant to the single therapy, but simultaneous use of SSI-4 inhibited the drug resistance in both models (FIGS. 2A and 2B). Tumor volumes were significantly lower in the groups of cabozantinib or lenvatinib, treated together SSI-4 treatment, compared to the single treatment (FIGS. 2C and 2D).

Together, these results demonstrate that one or more SCD1 polypeptide inhibitors (e.g., a SSI such as SSI-4) and one or more tyrosine kinase inhibitors (e.g., cabozantinib and/or lenvatinib) can act synergistically to treat cancer (e.g., liver cancer such as HCC).

### Example 2: SCD1 Polypeptide Inhibitors and Tyrosine Kinase Inhibitors for Treating HCC

This Example describes the anti-tumor synergy of one or more SCD1 polypeptide inhibitors (e.g., SSI-4, SSI-2, MF-438, and/or A939572) and one or more tyrosine kinase inhibitors (e.g., cabozantinib and/or lenvatinib) resulting in enhanced tumor regression (e.g., as compared to treating with only a single agent).

The results in this Example re-present and expand on at least some of the results provided in other Examples.

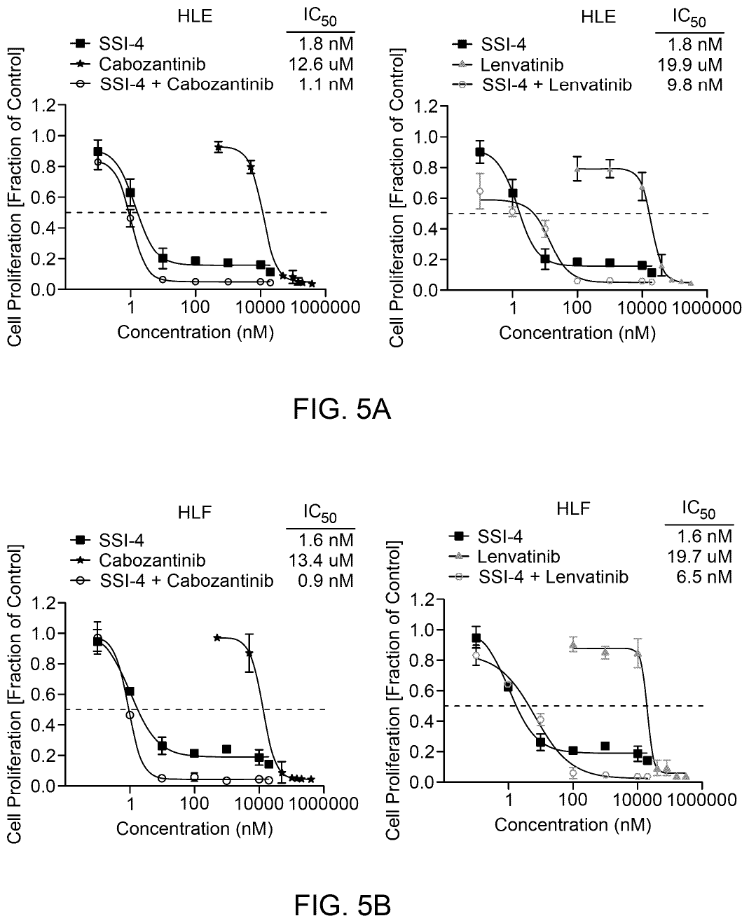

TABLE 4
IC50 values for combination treatments shown in FIG. 5A.
CI For experimental values HLE

| SSI-4 | Cabozantinib | | | |
|---|---|---|---|---|
| (nM) | (nM) | Fa | CI | Effect |
| 0.1 | 500 | 0.166 | 2.008 | antagonism |
| 1 | 5000 | 0.535 | 0.542 | synergism |
| 10 | 50000 | 0.938 | 0.347 | synergism |
| 100 | 100000 | 0.941 | 0.660 | synergism |
| 1000 | 150000 | 0.950 | 0.832 | synergism |
| 10000 | 200000 | 0.953 | 1.113 | antagonism |
| 20000 | 400000 | 0.956 | 2.052 | antagonism |

CI For experimental values HLE

| SSI-4 | Lenvatinib | | | |
|---|---|---|---|---|
| (nM) | (nM) | Fa | CI | Effect |
| 0.1 | 100 | 0.354 | 0.131 | synergism |
| 1 | 1000 | 0.488 | 0.384 | synergism |
| 10 | 10000 | 0.601 | 1.597 | antagonism |
| 100 | 40000 | 0.354 | 0.241 | synergism |
| 1000 | 80000 | 0.940 | 0.515 | synergism |
| 10000 | 160000 | 0.943 | 0.751 | synergism |
| 20000 | 320000 | 0.945 | 1.376 | antagonism |

TABLE 5
IC50 values for combination treatments shown in FIG. 5B.
CI For experimental values HLE

| SSI-4 | Cabozantinib | | | |
|---|---|---|---|---|
| (nM) | (nM) | Fa | CI | Effect |
| 0.1 | 500 | 0.028 | 3675.8 | antagonism |
| 1 | 5000 | 0.533 | 0.384 | synergism |
| 10 | 50000 | 0.949 | 0.311 | synergism |
| 100 | 100000 | 0.941 | 0.706 | synergism |
| 1000 | 150000 | 0.965 | 0.656 | synergism |
| 10000 | 200000 | 0.957 | 1.073 | antagonism |
| 20000 | 400000 | 0.962 | 1.899 | antagonism |

CI For experimental values HLE

| SSI-4 | Lenvatinib | | | |
|---|---|---|---|---|
| (nM) | (nM) | Fa | CI | Effect |
| 0.1 | 100 | 0.164 | 2.838 | antagonism |
| 1 | 1000 | 0.350 | 0.978 | synergism |
| 10 | 10000 | 0.589 | 1.183 | antagonism |
| 100 | 40000 | 0.940 | 0.184 | synergism |
| 1000 | 80000 | 0.953 | 0.261 | synergism |
| 10000 | 160000 | 0.964 | 0.374 | synergism |
| 20000 | 320000 | 0.963 | 0.777 | synergism |

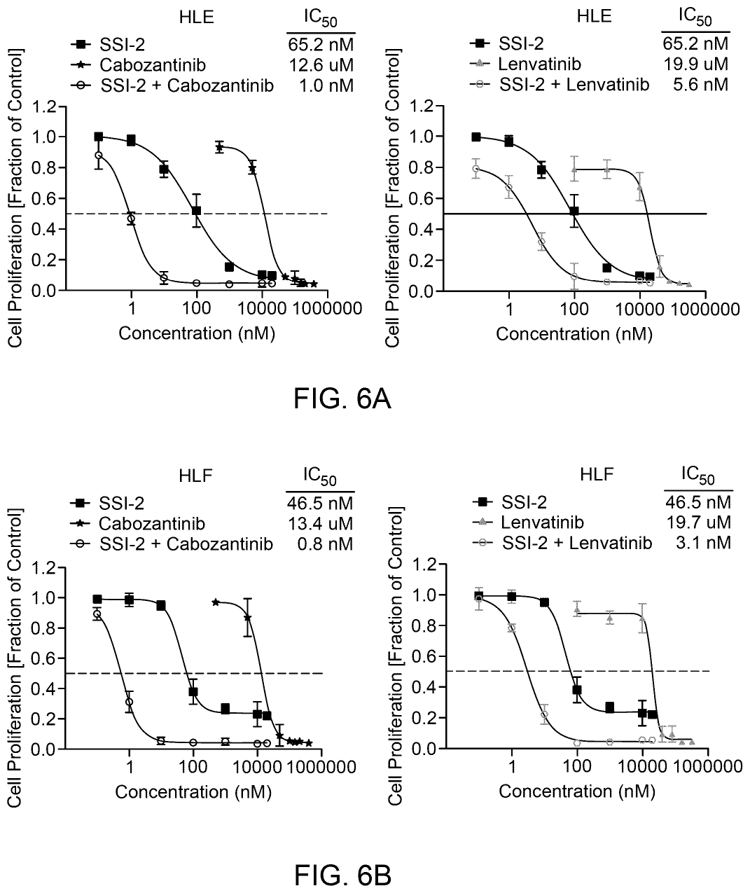

TABLE 6
IC50 values for combination treatments shown in FIG. 6A.
CI For experimental values HLE

| SSI-2 | Cabozantinib | | | |
|---|---|---|---|---|
| (nM) | (nM) | Fa | CI | Effect |
| 0.1 | 500 | 0.121 | 0.424 | synergism |
| 1 | 5000 | 0.531 | 0.483 | synergism |
| 10 | 50000 | 0.917 | 0.476 | synergism |
| 100 | 100000 | 0.945 | 0.614 | synergism |
| 1000 | 150000 | 0.958 | 0.716 | synergism |
| 10000 | 200000 | 0.949 | 1.546 | antagonism |
| 20000 | 400000 | 0.953 | 2.752 | antagonism |

CI For experimental values HLE

| SSI-2 | Lenvatinib | | | |
|---|---|---|---|---|
| (nM) | (nM) | Fa | CI | Effect |
| 0.1 | 100 | 0.205 | 0.185 | synergism |
| 1 | 1000 | 0.325 | 0.725 | synergism |
| 10 | 10000 | 0.677 | 0.799 | synergism |
| 100 | 40000 | 0.901 | 0.361 | synergism |
| 1000 | 80000 | 0.934 | 0.425 | synergism |
| 10000 | 160000 | 0.934 | 1.372 | antagonism |
| 20000 | 320000 | 0.946 | 1.966 | antagonism |

TABLE 7
IC50 values for combination treatments shown in FIG. 6B.
CI For experimental values HLE

| SSI-2 | Cabozantinib | | | |
|---|---|---|---|---|
| (nM) | (nM) | Fa | CI | Effect |
| 0.1 | 500 | 0.103 | 0.258 | synergism |
| 1 | 5000 | 0.686 | 0.198 | synergism |
| 10 | 50000 | 0.945 | 0.331 | synergism |
| 100 | 100000 | 0.951 | 0.602 | synergism |
| 1000 | 150000 | 0.950 | 0.927 | synergism |
| 10000 | 200000 | 0.965 | 0.966 | synergism |
| 20000 | 400000 | 0.961 | 2.126 | antagonism |

CI For experimental values HLE

| SSI-2 | Lenvatinib | | | |
|---|---|---|---|---|
| (nM) | (nM) | Fa | CI | Effect |
| 0.1 | 100 | 0.025 | 2.000 | antagonism |
| 1 | 1000 | 0.214 | 0.905 | synergism |
| 10 | 10000 | 0.778 | 0.324 | synergism |
| 100 | 40000 | 0.963 | 0.097 | synergism |
| 1000 | 80000 | 0.957 | 0.243 | synergism |
| 10000 | 160000 | 0.946 | 0.795 | synergism |
| 20000 | 320000 | 0.946 | 1.569 | antagonism |

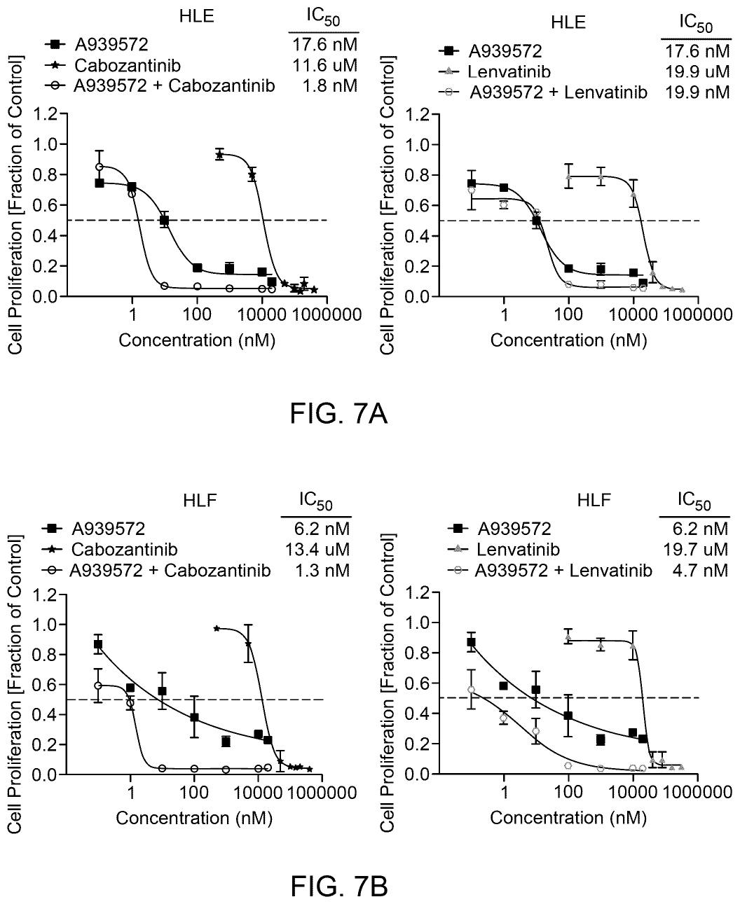

TABLE 8
IC50 values for combination treatments shown in FIG. 7A.
CI For experimental values HLE

| A939572 | Cabozantinib | | | |
|---|---|---|---|---|
| (nM) | (nM) | Fa | CI | Effect |
| 0.1 | 500 | 0.150 | 44.88 | antagonism |
| 1 | 5000 | 0.331 | 7.557 | antagonism |
| 10 | 50000 | 0.932 | 0.378 | synergism |
| 100 | 100000 | 0.936 | 0.713 | synergism |
| 1000 | 150000 | 0.944 | 0.938 | synergism |
| 10000 | 200000 | 0.952 | 1.072 | antagonism |
| 20000 | 400000 | 0.955 | 1.967 | antagonism |

CI For experimental values HLE

| A939572 | Lenvatinib | | | |
|---|---|---|---|---|
| (nM) | (nM) | Fa | CI | Effect |
| 0.1 | 100 | 0.299 | 1.258 | antagonism |
| 1 | 1000 | 0.395 | 2.494 | antagonism |
| 10 | 10000 | 0.443 | 12.52 | antagonism |
| 100 | 40000 | 0.919 | 0.251 | synergism |
| 1000 | 80000 | 0.921 | 0.501 | synergism |
| 10000 | 160000 | 0.938 | 0.697 | synergism |
| 20000 | 320000 | 0.945 | 1.143 | antagonism |

TABLE 9
IC50 values for combination treatments shown in FIG. 7B.
CI For experimental values HLE

| A939572 | Cabozantinib | | | |
|---|---|---|---|---|
| (nM) | (nM) | Fa | CI | Effect |
| 0.1 | 500 | 0.407 | 0.071 | synergism |
| 1 | 5000 | 0.522 | 0.382 | synergism |
| 10 | 50000 | 0.959 | 0.255 | synergism |
| 100 | 100000 | 0.963 | 0.467 | synergism |
| 1000 | 150000 | 0.965 | 0.664 | synergism |
| 10000 | 200000 | 0.957 | 1.066 | antagonism |
| 20000 | 400000 | 0.955 | 2.221 | antagonism |

CI For experimental values HLE

| A939572 | Lenvatinib | | | |
|---|---|---|---|---|
| (nM) | (nM) | Fa | CI | Effect |
| 0.1 | 100 | 0.507 | 0.019 | synergism |
| 1 | 1000 | 0.628 | 0.086 | synergism |
| 10 | 10000 | 0.717 | 0.496 | synergism |
| 100 | 40000 | 0.946 | 0.159 | synergism |
| 1000 | 80000 | 0.967 | 0.164 | synergism |
| 10000 | 160000 | 0.963 | 0.387 | synergism |
| 20000 | 320000 | 0.964 | 0.738 | synergism |

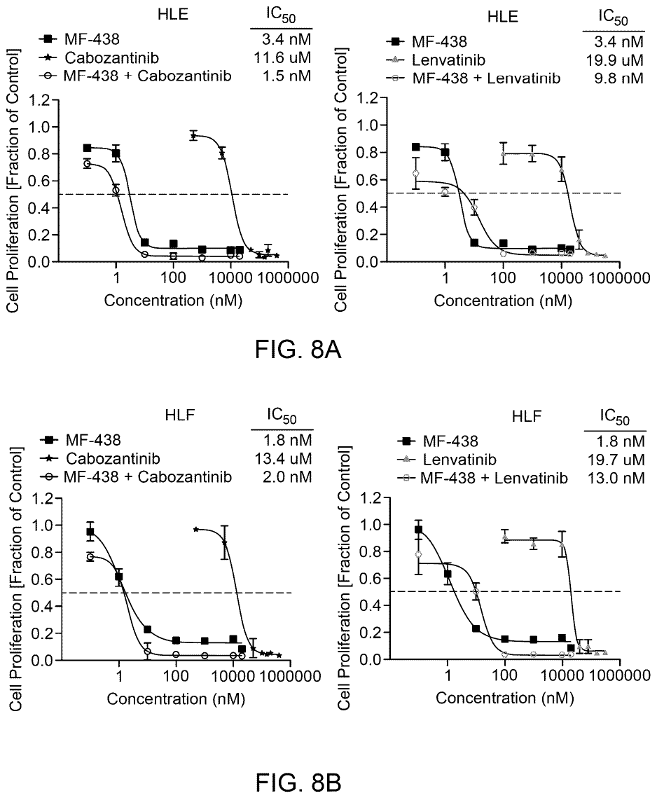

TABLE 10
IC50 values for combination treatments shown in FIG. 8A.
CI For experimental values HLE

| MF-438 | Cabozantinib | | | |
|---|---|---|---|---|
| (nM) | (nM) | Fa | CI | Effect |
| 0.1 | 500 | 0.275 | 0.652 | synergism |
| 1 | 5000 | 0.472 | 1.020 | additive |
| 10 | 50000 | 0.945 | 0.302 | synergism |
| 100 | 100000 | 0.961 | 0.424 | synergism |
| 1000 | 150000 | 0.971 | 0.466 | synergism |
| 10000 | 200000 | 0.952 | 1.546 | antagonism |
| 20000 | 400000 | 0.959 | 2.463 | antagonism |

CI For experimental values HLE

| MF-438 | Lenvatinib | | | |
|---|---|---|---|---|
| (nM) | (nM) | Fa | CI | Effect |
| 0.1 | 100 | 0.354 | 0.233 | synergism |
| 1 | 1000 | 0.488 | 0.605 | synergism |
| 10 | 10000 | 0.601 | 2.219 | antagonism |
| 100 | 40000 | 0.940 | 0.166 | synergism |
| 1000 | 80000 | 0.940 | 0.416 | synergism |
| 10000 | 160000 | 0.943 | 1.481 | antagonism |
| 20000 | 320000 | 0.945 | 2.651 | antagonism |

TABLE 11
IC50 values for combination treatments shown in FIG. 8B.
CI For experimental values HLE

| MF-438 | Cabozantinib | | | |
|---|---|---|---|---|
| (nM) | (nM) | Fa | CI | Effect |
| 0.1 | 500 | 0.231 | 0.392 | synergism |
| 1 | 5000 | 0.386 | 0.947 | synergism |
| 10 | 50000 | 0.932 | 0.404 | synergism |
| 100 | 100000 | 0.954 | 0.570 | synergism |
| 1000 | 150000 | 0.965 | 0.675 | synergism |
| 10000 | 200000 | 0.964 | 1.003 | additive |
| 20000 | 400000 | 0.967 | 1.867 | antagonism |

CI For experimental values HLE

| MF-438 | Lenvatinib | | | |
|---|---|---|---|---|
| (nM) | (nM) | Fa | CI | Effect |
| 0.1 | 100 | 0.225 | 0.394 | synergism |
| 1 | 1000 | 0.369 | 0.778 | synergism |
| 10 | 10000 | 0.500 | 2.647 | antagonism |
| 100 | 40000 | 0.967 | 0.081 | synergism |
| 1000 | 80000 | 0.961 | 0.215 | synergism |
| 10000 | 160000 | 0.968 | 0.383 | synergism |
| 20000 | 320000 | 0.964 | 0.948 | synergism |

### Example 3: Treating Cancer

A human identified as having cancer (e.g., liver cancer such as HCC) is administered (a) one or more SCD1 polypeptide inhibitors (e.g., a SSI such as SSI-4) and (b) one or more tyrosine kinase inhibitors (e.g., cabozantinib and/or lenvatinib). The administered combination can reduce the number of cancer cells present within the human.

### Example 4: Treating Cancer

A human identified as having cancer (e.g., liver cancer such as HCC) is administered (a) one or more SCD1 polypeptide inhibitors (e.g., a SSI such as SSI-4) and (b) one or more tyrosine kinase inhibitors (e.g., cabozantinib and/or lenvatinib). The administered combination can reduce the volume of one or more tumors present within the human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating cancer in a mammal, wherein said method comprises administering, to said mammal, a stearoyl CoA desaturase 1 (SCD1) polypeptide inhibitor and a tyrosine kinase inhibitor, wherein the number of cancer cells within said mammal is reduced.

2. The method of claim 1, wherein said mammal is a human.

3. The method of claim 1, wherein said cancer is a liver cancer.

4. The method of claim 1, wherein said SCD1 polypeptide inhibitor is a compound having Formula (II) or Formula (IIa): 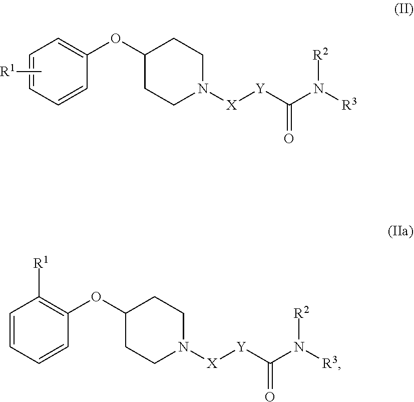
or a pharmaceutically acceptable salt thereof;
wherein:
R¹ is halo;
X is —(C═O)NR⁴—;
Y is 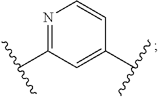 [SMILES: *c1ccnc(*)c1]
and
R², R³, and R⁴ are each independently H or an unsubstituted C₁₋₆ alkyl.

5. The method of claim 4, wherein said SCD1 polypeptide inhibitor is SSI-4, 2-{[4-(2-Chlorophenoxy)piperidine-1-carbonyl]amino}-N-methylpyridine-4-carboxamide: 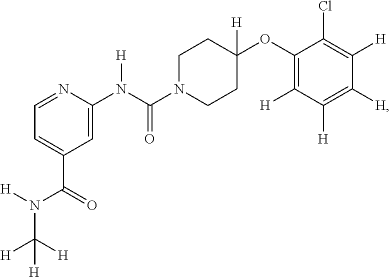
or a pharmaceutically acceptable salt thereof.

6. The method of claim 1, wherein said SCD1 polypeptide inhibitor is selected from the group consisting of: SSI-2 (2-(benzyloxy)-5-{[hydroxy({4-[2-(trifluoromethyl)benzoyl]piperazin-1-yl})methyl]amino}-1,2-dihydropyridin-2-ylium-1-ide) 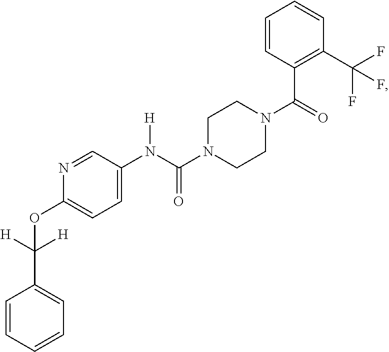 [SMILES: O=C(Nc1ccc(OCc2ccccc2)nc1)N1CCN(C(=O)c2ccccc2C(F)(F)F)CC1]
or a pharmaceutically acceptable salt thereof,
MF-438 (2-Methyl-5-(6-(4-(2-trifluoromethyl)phenoxy)piperidin-3-yl)-1,3,4-thiadiazole) 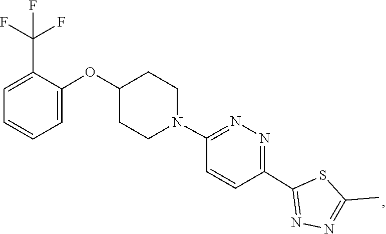 [SMILES: Cc1nnc(-c2ccc(N3CCC(Oc4ccccc4C(F)(F)F)CC3)nn2)s1]
or a pharmaceutically acceptable sale thereof, and A939572 (4-(2-Chlorophenoxy)-N-[3-(methylcarbamoyl)phenyl]piperidine-1-carboxamide) 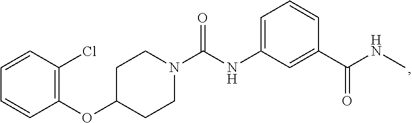 [SMILES: CNC(=O)c1cccc(NC(=O)N2CCC(Oc3ccccc3Cl)CC2)c1]
or a pharmaceutically acceptable sale thereof.

7. The method of claim 1, wherein said tyrosine kinase inhibitor inhibits one or more polypeptides selected from the group consisting of a vascular endothelial growth factor receptor, a platelet-derived growth factor receptor, a fibroblast growth factor receptor, a receptor tyrosine kinase encoded by a c-KIT proto-oncogene, a receptor tyrosine kinase encoded by a RET proto-oncogene, a hepatocyte growth factor receptor, a receptor tyrosine kinase encoded by an AXL proto-oncogene, a FMS like tyrosine kinase 3, a tropomyosin receptor kinase B, and a TIE-2.

8. The method of claim 1, wherein said tyrosine kinase inhibitor is selected from the group consisting of cabozantinib, lenvatinib, sorafenib, sunitinib, and regorafenib.

9. The method of claim 1, wherein said method comprises administering two or more SCD1 polypeptide inhibitors to said mammal.

10. The method of claim 1, wherein said method comprises administering two or more tyrosine kinase inhibitors to said mammal.

11. A method for treating cancer in a mammal, wherein cancer cells of said cancer have resistance to treatment with a tyrosine kinase inhibitor alone, wherein said method comprises administering, to said mammal, a SCD1 polypeptide and said tyrosine kinase inhibitor, wherein the number of cancer cells within said mammal is reduced.

12. The method of claim 11, wherein said method comprises determining that said cancer cells are resistant to treatment with said tyrosine kinase inhibitor alone.

13. The method of claim 11, wherein said mammal is a human.

14. The method of claim 11, wherein said cancer is a liver cancer.

15. The method of claim 11, wherein said SCD1 polypeptide inhibitor is a compound having Formula (II) or Formula (IIa): 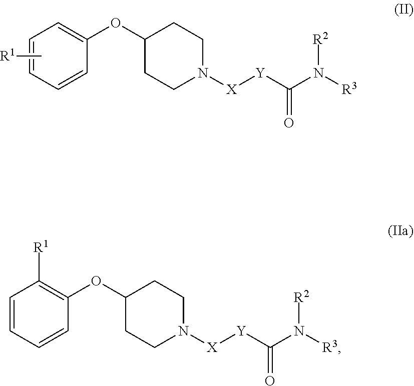
or a pharmaceutically acceptable salt thereof;
wherein:
R¹ is halo;
X is —(C═O)NR⁴—;
Y is 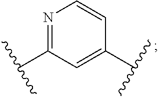 [SMILES: *c1ccnc(*)c1]
and
R², R³, and R⁴ are each independently H or an unsubstituted C₁₋₆ alkyl.

16. The method of claim 15, wherein said SCD1 polypeptide inhibitor is SSI-4, 2-{[4-(2-Chlorophenoxy)piperidine-1-carbonyl]amino}-N-methylpyridine-4-carboxamide: 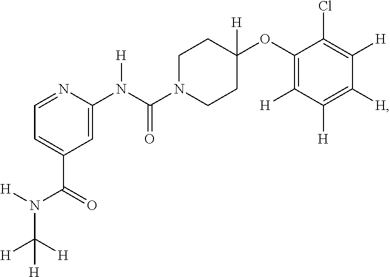
or a pharmaceutically acceptable salt thereof.

17. The method of claim 11, wherein said SCD1 polypeptide inhibitor is selected from the group consisting of: SSI-2 (2-(benzyloxy)-5-{[hydroxy({4-[2-(trifluoromethyl)benzoyl]piperazin-1-yl})methyl]amino}-1,2-dihydropyridin-2-ylium-1-ide) 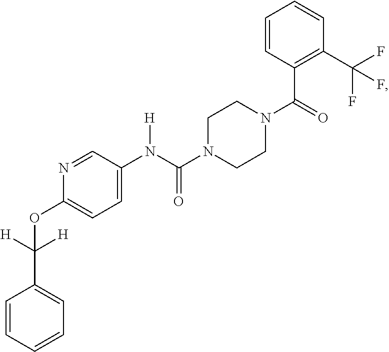 [SMILES: O=C(Nc1ccc(OCc2ccccc2)nc1)N1CCN(C(=O)c2ccccc2C(F)(F)F)CC1]
or a pharmaceutically acceptable salt thereof,
MF-438 (2-Methyl-5-(6-(4-(2-trifluoromethyl)phenoxy)piperidin-3-yl)-1,3,4-thiadiazole) 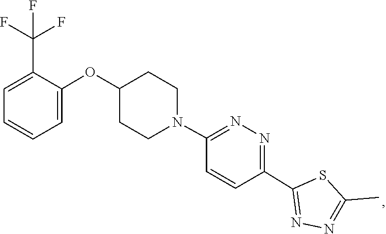 [SMILES: Cc1nnc(-c2ccc(N3CCC(Oc4ccccc4C(F)(F)F)CC3)nn2)s1]
or a pharmaceutically acceptable sale thereof, and
A939572 (4-(2-Chlorophenoxy)-N-[3-(methylcarbamoyl)phenyl]piperidine-1-carboxamide) 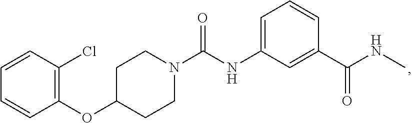 [SMILES: CNC(=O)c1cccc(NC(=O)N2CCC(Oc3ccccc3Cl)CC2)c1]
or a pharmaceutically acceptable sale thereof.

18. The method of claim 11, wherein said tyrosine kinase inhibitor inhibits one or more polypeptides selected from the group consisting of a vascular endothelial growth factor receptor, a platelet-derived growth factor receptor, a fibroblast growth factor receptor, a receptor tyrosine kinase encoded by a c-KIT proto-oncogene, a receptor tyrosine kinase encoded by a RET proto-oncogene, a hepatocyte growth factor receptor, a receptor tyrosine kinase encoded by an AXL proto-oncogene, a FMS like tyrosine kinase 3, a tropomyosin receptor kinase B, and a TIE-2.

19. The method of claim 11, wherein said tyrosine kinase inhibitor is selected from the group consisting of cabozantinib, lenvatinib, sorafenib, sunitinib, and regorafenib.

20. The method of claim 11, wherein said method comprises administering two or more SCD1 polypeptide inhibitors to said mammal.

21. The method of claim 11, wherein said method comprises administering two or more tyrosine kinase inhibitors to said mammal.
